(19) United States
(12) Patent Application Publication
(10) Pub. No.: US 2006/0147941 A1
(43) Pub. Date: Jul. 6, 2006

(54) Methods and apparatus for SERS assay of biological analytes

(75) Inventor: Xing Su, Cupertino, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 11/026,857

(22) Filed: Dec. 30, 2004

(51) Int. Cl. C12Q 1/68

(52) U.S. Cl. 435/6; 977/924

(57) ABSTRACT

SERS technology is used for high throughput screening of biological analytes and samples. For polynucleotide sequencing, sets of oligonucleotide probes are labeled with composite organic-inorganic nanoparticles (COIN) that produce distinguishable SERS signals when excited by a laser. Detection of a hybridization complex containing members of two such COIN-labeled probe sets will reveal a 12 nucleotide sequence segment of the target polynucleotide. Also provided are surface-modified arrays and chips with multiple arrays to which sets of probe-conjugated COIN or other reporter substrates are immobilized. Analytes are detected by contacting a sample, such as a bodily fluid, with the array-anchored probes. Captured analytes are tagged with an additional target-specific Raman-active tag. Two or more Raman signatures emanating from the detection complexes reveal the identity of the captured analytes.

### BACKGROUND OF THE INVENTION

1. Field of the Invention

The invention relates generally to nanoparticles that include metallic colloids and organic compounds, and more specifically to the use of such nanoparticles in analyte detection by surface-enhanced Raman spectroscopy.

2. Background Information

Multiplex reactions are parallel processes that exist naturally in the physical and biological worlds. When this principle is applied to increase efficiencies of biochemical or clinical analyses, the principal challenge is to develop a probe identification system that has distinguishable components for each individual probe in a large probe set. High-density DNA chips and microarrays are probe identification systems in which physical positions on a solid surface are used to identify nucleic acid or protein probes. The method of using striped metal bars as nanocodes for probe identification in multiplex assays is based on images of the metal physical structures. Quantum dots are particle-size-dependent fluorescent emitting complexes.

Biochips, including DNA arrays (DNA chips), microarrays, protein arrays, and the like, are devices that may be used to perform highly parallel biochemical reactions. Such devices have been fabricated either by building the biomolecules (nucleic acids or proteins) as probes on the chip surface directly or depositing the biomolecules on the chip surface after they have been synthesized. Generally physical positions (XY coordinates) are used to identify the properties or sequences of detected probes molecules.

The ability to detect and identify trace quantities of analytes has become increasingly important in virtually every scientific discipline, ranging from part per billion analyses of pollutants in sub-surface water to analysis of cancer treatment drugs in blood serum. Raman spectroscopy is one analytical technique that provides rich optical-spectral information, and surface-enhanced Raman spectroscopy (SERS) has proven to be one of the most sensitive methods for performing quantitative and qualitative analyses. A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Among many analytical techniques that may be used for chemical structure or nucleotide sequence analysis, Raman spectroscopy is attractive for its capability in providing rich structure information from a small optically focused area or detection cavity. Compared to a fluorescent spectrum that normally has a single peak with half peak width of tens of nanometers (quantum dots) to hundreds of nanometers (fluorescent dyes), a Raman spectrum has multiple bonding-structure-related peaks with half peak width of as small as a few nanometers. Furthermore, surface enhanced Raman scattering (SERS) techniques make it possible to obtain a 10⁶ to 10¹⁴ fold Raman signal enhancement, and may even allow for single molecule detection sensitivity. Such huge enhancement factors may be attributed primarily to enhanced electromagnetic fields on curved surfaces of coinage metals. Although the electromagnetic enhancement (EME) has been shown to be related to the roughness of metal surfaces or particle size when individual metal colloids are used, SERS is most effectively detected from aggregated colloids. It is known that chemical enhancement may also be obtained by placing molecules in a close proximity to the surface in certain orientations. Due to the rich spectral information and sensitivity, Raman signatures have been used as probe identifiers to detect a few attomoles of molecules when SERS method was used to boost the signals of specifically immobilized Raman label molecules, which in fact are the direct analytes of the SERS reaction. The method of attaching metal particles to Raman-label-coated metal particles to obtain SERS-active complexes has also been studied. A recent study demonstrated that a SERS signal may be generated after attachment of thiol containing dyes to gold particles followed silica coating.

Analyses for numerous chemicals and biochemicals by SERS have been demonstrated using: (1) activated electrodes in electrolytic cells; (2) activated silver and gold colloid reagents; and (3) activated silver and gold substrates.

SERS technique may identify and detect single molecules without labeling. SERS effect is attributed mainly to electromagnetic field enhancement and chemical enhancement. It has been reported that silver particle sizes within the range of 50-100 nm are most effective for SERS. Theoretical and experimental studies also reveal that metal particle junctions are the sites for efficient SERS.

### DESCRIPTION OF THE FIGURES

The drawings accompanying and forming part of this specification are included to depict certain aspects of embodiments of the invention. A clearer conception of the embodiments of the invention, and of the components and operation of systems provided with embodiments of the invention, will become more readily apparent by referring to the exemplary, and therefore non-limiting, embodiments illustrated in the drawings, wherein identical reference numerals designate the same elements. The embodiments of the invention may be better understood by reference to one or more of these drawings in combination with the description presented herein. It should be noted that the features illustrated in the drawings are not necessarily drawn to scale. Brief descriptions are provided below, followed a detailed description of the preferred embodiments in view of the illustrative drawings.

FIG. 1A is a flow diagram illustrating the concept of the invention methods for using composite organic-inorganic nanoparticles (COIN) to sequence a six-nucleotide segment of a polynucleotide. FIG. 1B is an illustrative drawing showing a reporter-substrate (RS) set for use with nanoparticles of the invention.

FIG. 2 is a schematic drawing illustrating of a probe-COIN conjugate attached to an array surface.

FIG. 3 is a drawing of an invention chip containing a 4×4 array (16 subarrays) useful for fully sequencing a nucleic acid containing 1.6×10⁷ nucleotides using invention methods and systems.

FIG. 4 is a flow chart illustrating the sequencing of a polynucleotide using invention methods

FIGS. 5A and 5B illustrate two types of array arrangement: regular array FIG. 5A and non-regular array (FIG. 5B).

FIG. 6 is a schematic drawing illustrating modification of array adhere surfaces in invention arrays with surface attachment coupling agents that present a free functional group for coupling with a binding partner that will form a specific binding pair with a binding partner on a reporter substrate. FIG. 6 illustrates a gold adhere surface modified with a compound that presents a free thiol group or a glass adhere surface modified with a compound that presents a free silane group.

FIGS. 7A, 7B, and 7C are a series of schematic drawings illustrating three different specific binding partners used to immobilize a reporter substrate to an array adhere surface. FIG. 7A shows an antibody probe binding with a Protein G or Protein A modified surface; FIG. 7B shows a Poly(dA) modified reporter substrate binding with a poly(T) modified surface; and FIG. 7C shows a biotin modified reporter substrate binding with a strepavidin modified surface.

FIGS. 8A and 8B illustrate additional types of subarray formats on a chip: a set of subarrays on a flat surface (FIG. 8A) and columnar subarrays formed in fluid channels.

FIGS. 9A and 9B are schematic drawings illustrating a one-step detection assay (FIG. 9A) and a two-step detection assay (FIG. 9B) utilizing probe-conjugated reporter substrates attached to an invention array.

FIGS. 10A and 10B are graphs showing SERS signatures of COINs made with individual (FIG. 10A) or mixtures (FIG. 10B) of Raman labels

FIGS. 11A and 11B are a diagram showing components of an apparatus for receiving, detecting or processing a Raman signal.

### DETAILED DESCRIPTION OF THE INVENTION

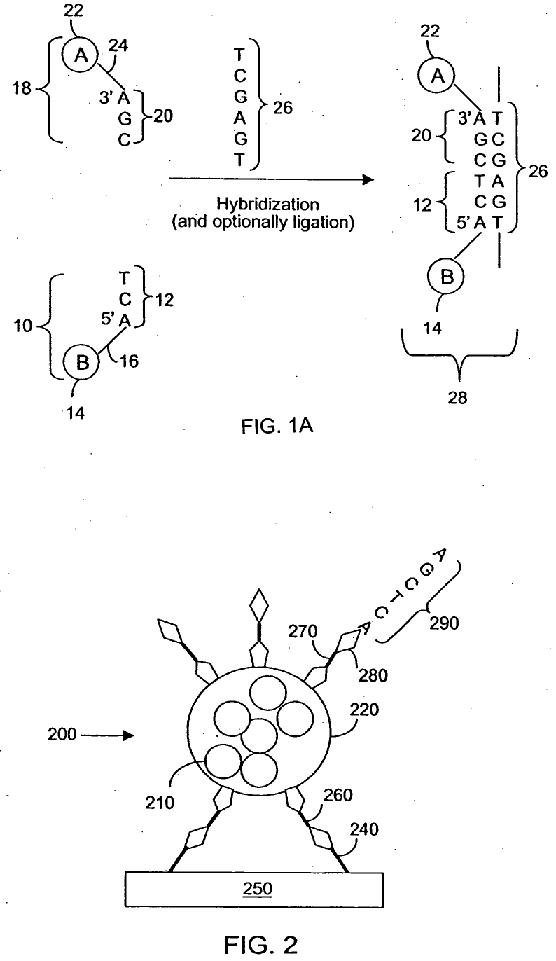

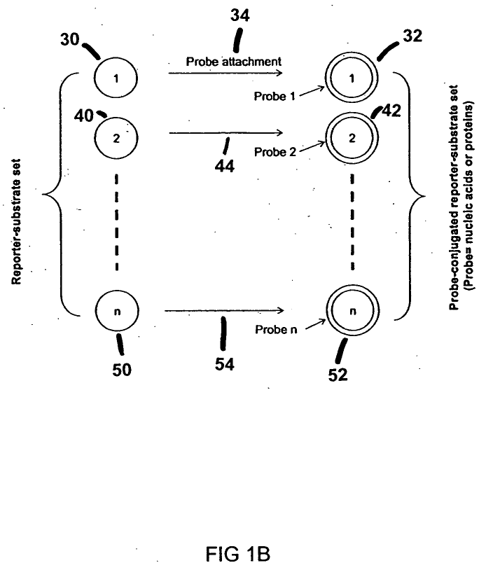

The general concept of the invention will now be described with reference to FIG. 1A and FIG. 1B, in which, for illustrative purposes. In one embodiment, the invention provides a system for sequencing a nucleic acid target molecule using two sets of composite organic-inorganic nanoparticles (COIN)-labeled probes, wherein the probes are oligonucleotide sequences of fixed length including at least a sextet sequence of nucleic acids, which is referred to herein as the “probe sequence”. One or more of the oligonucleotide probes are attached to a COIN (label), or a COIN-containing COIN bead as described herein. In a 5′ probe set, the probe oligonucleotide is attached to the COIN label via the 5′ end to leave a free 3′ end and in a 3′ probe set, the probe oligonucleotide is attached to the COIN label via the 3′ end to leave a 5′ end of the oligonucleotide probe free. In illustrative FIG. 1A, the probe sequences contain three nucleotides. A 5′ probe 10 has probe nucleotide sequence 12 (5′ ACT 3′) attached to a COIN label 14 via linker 16 leaving the 3′ end of the probe sequence free. A 3′ probe 18 has probe nucleotide sequence 20 (5′CGA3′) attached to a COIN label 22 via linker 24 leaving the 5′ end of the probe sequence free. Target sequence 26 (5′TCGAGT 3′) is contacted under specific hybridization conditions with the 5′ probe 12 and the 3′ probe 20. In the hybridization complex formed, 28, the presence of Raman signatures produced by both COIN labels 14 and 22 indicates the presence of the target sequence 26 in the sample. Probe sequences 12, 20 optionally may be ligated prior to detection. The COIN labels have specific Raman signatures indicating the known oligonucleotide sequences of the probes. A single COIN is about 100 nm in dimension and a COIN bead may be made to contain as many as 10 to 100 individual COINs, or more. In practice one or more probe nucleotide sequences may be attached to a single COIN or COIN bead.

FIG. 1B is an illustrative diagram of the Reporter-Substrate (RS) sets shown in FIG. 1A. A probe sequence 32, 42, 52 may be nucleic acid (e.g., DNA, RNA) or a protein (e.g., antibody, receptor), for example. A reporter 30, 40, 50 for producing an optical signal (e.g., Raman, fluorescence) for probe identification, and a substrate or material providing a surface for probe attachment (e.g., COIN) are an RS which has a dual function for probe attachment and identification. A probe sequence (e.g., 32) is linked to a COIN label (e.g., 30, 40, 50) via a linker 34, 44, 54 in FIG. 1B.

Methods for using composite organic-inorganic nanoparticles (COIN) to assay biological samples are provided herein and illustrated in FIG. 1A and 1B. The nanoparticles include several fused or aggregated primary metal crystal particles with Raman-active organic compounds adsorbed on the surface, in the junctions of the primary particles, or embedded in the crystal lattice of the primary metal particles. Any of the Raman-active organic compounds adsorbed on the exterior of the COIN are typically less Raman-active than if situated between metal surfaces or metal crystals.

Accordingly, in one embodiment, the invention provides a system for sequencing a polynucleotide. The system includes 1) one or more subsets of a first probe set, wherein a member of the first probe set includes one or more probes of at least about 3 nucleotides and at least one label to produce distinguishable first and second optical signatures, wherein the first optical signature indicates attachment orientation of the probes within the first probe set and the second optical signature is a Raman signature associated with a known probe sequence of the member within a subset of the first probe set, and 2) one or more subsets of a second probe set wherein a member of the second probe set includes one or more probes of at least about 3 nucleotides and at least one label to produce distinguishable third and fourth optical signatures, wherein the third optical signature indicates an attachment orientation of the probes to the label that is opposite to that of the first probe set and the fourth optical signature is a Raman signature associated with a known sequence of the oligonucleotides of the member within a subset of the second probe set, wherein the probe sequence of a member of a probe set is unique to the member within a respective probe set, and a probe set collectively includes all possible probe sequence combinations. Optionally, the system may further include one or more subsets of a third probe set, wherein a member of the third probe set is unlabelled, includes a probe of at least about 3 nucleotides, and forms a phosphodiester bond with a member of the first probe set. For example, the probe sequences may have a fixed length e.g., at least about 3 nucleotides. The first and third optical signatures may be fluorescent and the second and fourth optical signatures produced by using COINs as the labels, wherein the COIN labels in a probe set may produce as few as 100 or more distinguishable Raman signatures.

In one particular embodiment of the invention, the invention system may include one or more subsets of three different types of probe sets, referred to as first probe sets, second probe sets and third probe sets. Members of a first probe set include one or more identical oligonucleotide sequences of at least about 3 nucleotides, wherein the sequence is unique to the member within the first probe set, and a COIN label that produces first and second distinguishable optical signatures (for example, Raman or fluorescent signatures). The first optical signature indicates attachment orientation of the probes in the first probe sets and the second optical signature, which is Raman, is unique to a member within subset of the first probe set and is selected to indicate the probe sequence of the member.

In the third probe set, a member is unlabelled and includes an oligonucleotide sequence of at least about 3 nucleotides, wherein the oligonucleotide sequence is unique to the member within the second probe set.

In the second probe set, a member includes one or more identical oligonucleotide probes of at least about 3 nucleotides and in one aspect, at least about 6 nucleotides, wherein the probe is unique to the member within the second probe set, and a COIN label that produces distinguishable third and fourth Raman signatures. The third Raman signature indicates attachment orientation of the oligonucleotide probes to the label is opposite to that of the members of the first probe set and the fourth Raman signature is associated with the probe sequence of a member within a subset of the third probe set.

Attachment orientation of members of the first probe set may be either such as leaves a 3′ end of the probe sequence free or a 5′ end of the probe sequence free, but in either case all members of the first probe set must have the same attachment orientation and all members of third probe set must have attachment orientation opposite to that selected for the members of first probe set. Members of the second probe set, if present, are unlabelled, and all members of the second probe set are oriented during synthesis such that a member of a second probe set can form a phosphodiester bond with, or be ligated to, a member of a first probe set.

Although the nucleotide sequence of a member of a probe set is unique to the member within a respective first, second or third probe set, a probe set, whether a first, second or third probe set, collectively includes all possible probe sequence combinations and the set of probe sequences incorporated within a first, second or third probe set, therefore, is identical. The number of possible combinations is determined by the fixed number of nucleotides (e.g., 3 to 15) selected for use in the first and second (and optionally third) probe sequences, which must all contain the same fixed number of nucleotides. Additionally, the probes in the COIN-labeled probes may include zero to three additional degenerate nucleotides added at the labeled end to increase hybridization efficiency, for example by decreasing steric hindrance.

The number of different distinguishable Raman sequences used within a probe set may be as few as about 3 or more or as few as 100 and, in any event, can conveniently be determined by dividing the number of possible combinations in the probe set (determined by the fixed number of nucleotides selected for the probe sequences) by a whole integer to yield the number of different subsets of a probe set should be prepared so that members of a subset of a probe set all have distinguishable Raman signatures, with each subset containing an identical set of COINs. In other words, the whole integer may determine the number of subsets of any of the first, second and (if present) third probe sets prepared. These requirements are best explained with reference to a mathematical model. The model is based on the theory that the shortest oligonucleotide that perfectly and specifically binds to a complementary sequence under favorable hybridization conditions contains six nucleotides; hence fixed number of nucleotides used in the probe sequences in the model is 6 nucleotides. The mathematics for producing the first and third probe sets (those requiring COIN labels) are illustrated for the case wherein the oligonucleotide probes contain a sextet sequence that binds specifically to a complementary sequence in a target polynucleotide, or fragment thereof, as follows:

Probe length: 6 specific binding nucleotides, plus 0-3 optional degenerate nucleotides, making the oligonucleotide in a probe range from 6 to 9 nucleic acids in length

Probe orientations: 2 (a 3′ probe set oligonucleotide attaches to its label so as to have a free 5′ end; a 5′ probe set oligonucleotide attaches to its label so as to have a free 3′ end)

All possible sextet combinations for the two attachment orientations=2 orientations×(4 nucleic acids)ˆ6=2×4096 oligonucleotides per system

Length of genomic DNA covered by the system=4ˆ(6+6)=1.6×10ˆ7 nucleotides (this is about 1/200 of human genome covered)

No. of distinguishable COIN labels in a probe subset=1100, all with distinguishable SERS signatures, i.e., the whole integer used to divide the possible number of nucleotide combinations is 4.

Subsets of COIN labels per attachment orientation: 4096/1100=4, with each subset of probe-labeled COINs containing an identical set of COIN labels and the complete set containing all possible sextet combinations.

No. of arrays per chip@1.6×10ˆ7 nucleotides/array: 4×4 array=16 subarrays

Those of skill in the art will understand that, increasing the length of the probe sequences by even one additional nucleotide would require a much larger set of COIN labels to cover all the possible nucleotide combinations in a probe set, which may involve fewer than four copies of the first, second, and third probe sets. The set of COIN labels used in manufacture of the subsets of the first probe set may also be used in the making the subsets of the second probe sets, if coded with an additional detectable feature (for example an additional fluorescent or Raman-active organic compound) that distinguishes the first and second probe sets. An oligonucleotide probe may also contain an additional 1 to about 3 degenerate nucleotides (not targeting nucleotides) to facilitate hybridization reactions, for example at the end of the oligonucleotide that is attached to the COIN label. Methods for oligonucleotide synthesis are well known in the art and any such known method can be used. For example, oligonucleotides can be prepared using commercially available oligonucleotide synthesizers (for example, Applied Biosystems, Foster City, Calif.). Nucleotide precursors attached to a variety of tags can be commercially obtained (for example, from Molecular Probes, Eugene, Ore.) and incorporated into oligonucleotides or polynucleotides. Alternatively, nucleotide precursors can be purchased containing various reactive groups, such as biotin, diogoxigenin, sulfhydryl, amino or carboxyl groups. After oligonucleotide synthesis, tags can be attached using standard chemistries. Oligonucleotides of any desired sequence, with or without reactive groups for tag attachment, may also be purchased from a wide variety of sources (for example, Midland Certified Reagents, Midland, Tex.).

Probe-COIN label conjugation will now be described with reference to FIG. 2. COIN beads 200 may be used as the COIN label in fabrication of the first and third probe sets. In a COIN bead 200 several COIN particles 210 (each 50 to 200 nm in largest diameter) are embedded in a polymer bead 220 having a largest dimension of about 1 to about 5 microns in size, which is equivalent to a typical laser beam size of about 01. to about 10 microns, for example 1 to 5 microns. Surface attached coupling agent 240 on the surface of substrate 250, forms a specific binding pair with a functional group 260 on the polymer coating material of polymer bead 220. Linker molecule 270, also attached to the polymer coating material of polymer bead 220 using standard chemistry techniques, provides a cross-linking site 280 for conjugation of nucleotide probe 290 to linker molecule 270. In such COIN beads, a larger surface area than in COIN particles is available for attachment of nucleotide probes and much stronger Raman signals may be detected from a single COIN bead without losing detection resolution.

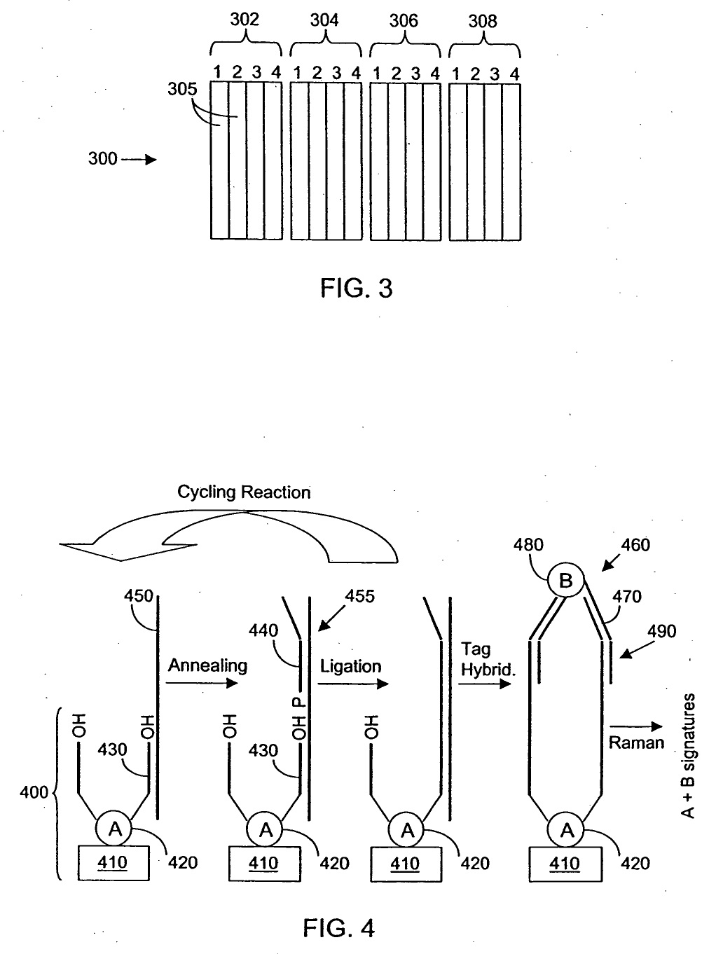

In the invention methods, the COIN-labeled oligonucleotide probes are used in a hybridization reaction to detect specific binding of certain of the COIN labeled oligonucleotide probes to a complementary target sextet oligonucleotide in solution. Alternatively, either the first or the third probe sets may be attached to a substrate surface for use. For example, as described with reference to FIG. 3 and based on the mathematical example of probe manufacture above, chip 300 has 16 columns 305, divided into four subarrays (302, 304, 306, 308), each subarray containing four of the columns. If a copy of a first probe set (for example, a copy of a 5′ probe set) is attached to fixed locations in each column (one copy per column) using methods known in the art and as described herein, the above calculations show that the 16 subarrays are sufficient to cover 1.6×10⁷ possible combinations of 12 nucleotide long target sequences of a target polynucleotide. Allowing for 10-fold redundancy for each type of COIN combination, there will be 1.6×10⁸ data points of sequence information obtained. If each data point requires 1 ms to scan and process, in 2 days one Raman reader can scan 1.6×10⁷ nucleotides. Therefore, this example illustrates that when a highly parallel photodiode array is used, the whole human genome may theoretically be sequenced in a few days using the invention methods, systems, and apparatus.

The method of using the invention system of probe sets to sequence a polynucleotide will now be described with reference to FIG. 4, which is a flow chart illustrating the invention methods wherein three probe sets are used to sequence a polynucleotide. FIG. 4 is a flow chart illustrating the sequencing of a polynucleotide using invention methods. A=sextet probe sequence with orientation of attachment to the COIN that leaves free the 3′ end of the sequence. B=sextet probe sequence with orientation of attachment to the COIN that leaves free the 5′ end of the sequence. A member 400 of a 5′ first probe set comprising COIN label 420 and probe sequence 430 is shown attached to a fixed location 410 on an array adhere surface. The two distinguishable optical signatures of COIN label 420 indicate 1) the sequence of attached probe sequence 430 (Raman signature) and 2) the attachment orientation of the probe sequence 430 as leaving the 3′ end of probe sequence 430 free (fluorescent or Raman signature). A reaction mixture includes the target polynucleotide 450 and a member of an unlabelled 3′ probe set 440 with a probe sequence having a free 5′ end, which hybridizes to the probe sequence 430 to form an unligated hybridization complex 455. Ligation reaction conditions may be introduced for ligation of the member 400 of the 5′ probe set and the member 440 of the unlabeled probe set contained in hybridization complex 455. These hybridization and ligation steps may be repeated until members of the first probe set and unlabeled probe set are depleted in the reaction mixture as shown in the cycling arrow in FIG. 4. Then the target molecule is removed and a 3′ probe set 460 whose members include probe sequence 470 and COIN label 480 are introduced to the reaction mixture and allowed to hybridize with the single stranded and complementary region of hybridization complex 455 to form tag hybridization complex 490. The two distinguishable optical signatures of COIN label 470 indicate 1) the sequence of attached probe sequence 430 (Raman signature) and 2) the attachment orientation of the probe sequence 470 (fluorescent or Raman signature) as leaving the 3′ end of probe sequence 470 free. In general, when three probe sets are used, as in FIG. 4, members of the probe sets 430 and 440 have 3′ ends free if the members of the unlabeled probe set 470 is to be ligated to the members of 430 probe set and vice versa. By contrast, when only two probe sets are used, as in FIG. 1, the members of the first and second probe sets have opposite attachment orientation so that opposite ends are free for ligation.

Detection of all four distinguishable optical signatures from a single fixed location 410 in an array indicates both formation at the fixed location of tag hybridization complex 490 and also the 12-nucleotide sequence of the double stranded segment of the target polynucleotide contained in the tag hybridization complex 490.

Optionally, in a ligation reaction, a member of a first probe set is ligated to a member of a probe set having opposite attachment orientation and contained in a hybridization complex to yield a 12 base targeting probe. As those of skill in the art will appreciate, ligation of members of the two probe sets in a hybridization complex may be accomplished when the attachment orientation of the two probe sequences is such that a free hydroxyl group on one and a free phosphate group on the other can combine to form a phosphodiester bond. Therefore, as used herein, in one or more embodiments of the inventionthe phrase “opposite orientation to the members of the first probe set” maymean that the second probe in the hybridization complex hybridizes in an orientation that provides the free moiety needed to form a phosphodiester bond with a member of the first probe set. However, the scope of the invention is not limted in this respect, and other definitions may be contemplated within the scope of the invention.

As a result, ligation may occur when the two probes involved form a perfect probe-target hybridization complex (two probes and one single stranded and complementary target sequence perfectly aligned and ligated, without mismatch) and ligated probes will have a 12 base sequence, for example. Ligated probes can be retained in a tag hybridization complex so formed when the target sequence is removed (by heating, in low ionic strength solution or in high pH). As COIN labels may have more than one nucleotide probe attached, the hybridization complex and tag hybridization complex may be stably held together by hybridization of several molecules.

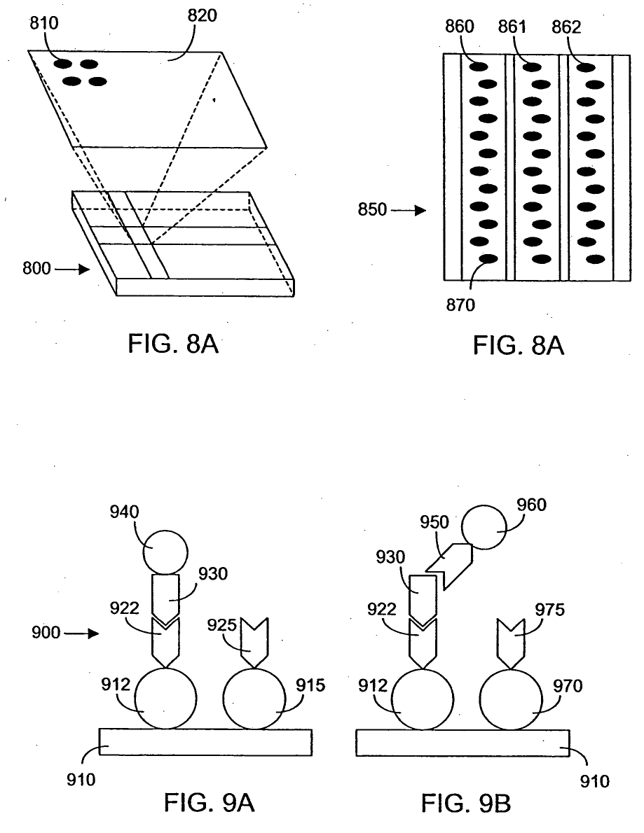

The contacting and the ligating steps in the method are repeated under thermocycling conditions until the second probe set and the third probe set are substantially depleted. Typical thermocycling conditions may include, for example, 40 cycles of incubation for 1 s at 93° C., 1 s at 59° C., and 1 min 10 s at 62° C. (see Journal of Clinical Microbiology (1998) 36(4):1028-1031). Microfluidic techniques may be used to control the reactions, for example on a chip containing multiple arrays comprising fluid channels, as illustrated in FIG. 8 herein.

The Raman signatures of captured COIN labeled oligonucleotide probes may be detected using Raman spectroscopy, with or without first being released from the fixed location on the array. Collection and assembly of Raman signature information provided by using the invention system for sequencing a polynucleotide may thus determine the sequence of a target polynucleotide target. Such a method is useful, for example, for sequencing of infectious agents within a clinical sample, sequencing an amplification product derived from genomic DNA or RNA or message RNA, or sequencing a gene (cDNA) insert within a clone.

In yet another embodiment, the invention provides arrays such as illustrated in FIG. 3, 5 and 8, for use in high throughput assays using a set of probe molecules conjugated to a set of reporter-substrates, such as a set of COIN-labeled probes as described herein and illustrated in FIG. 1 and FIG. 2. The reporter-substrates (RS) serve both as substrate for conjugation of a known probe molecule and as reporter molecules, the conjugate is referred to herein as a “probe-conjugated reporter substrate” or reporter substrate. An example of a probe-conjugated reporter substrate is a COIN-labeled probe, as described herein. A member of a set of the probe-conjugated reporter substrates produces an optical signal that is unique within the set, and is associated with the known probe molecule to which the reporter substrate is conjugated. Thus, the requirement to have arrayed probe molecules either built up while attached to an array substrate (so that the sequence is known) or deposited on the array at known addressable locations so that physical location (for example, XY coordinates on the array) may be used to identify the arrayed sequences or molecule properties, as in so-called “DNA chips,” is eliminated. Any probe molecule (such as an antibody, a receptor, an aptamer, RNA or DNA) that forms a specific binding pair with a desirable target biomolecule, including a protein, may be used as the probe in sets of probe-conjugated reporter substrates. Examples of reporter-substrates that can be used with the invention arrays in performance of the invention methods include, but are not limited to the COIN labels and COIN beads described herein as well as commercially available Luminex™ fluorescent beads (Luminex Corp., Austin, Tex.).

There are many techniques known in the art for conjugating a biomolecule to a solid support that may be applied to conjugation of a probe molecule to a reporter-substrate to form the probe-conjugated reporter substrates used with the invention arrays. For example and without limitation, amino groups on protein or nucleic acid probes may be attached to a reporter substrate, such as one or more COIN particles or COIN beads where there are available carboxyl groups through EDAC (1-ethyl-3-(3-dimethylaminopropyl) carbodiimide) chemistry. Using this technique, poly(dA) molecules, biotin, or probes, such as antibodies or oligonucleotides may be conjugated to carboxyl groups on these solid support surfaces (See FIG. 2).

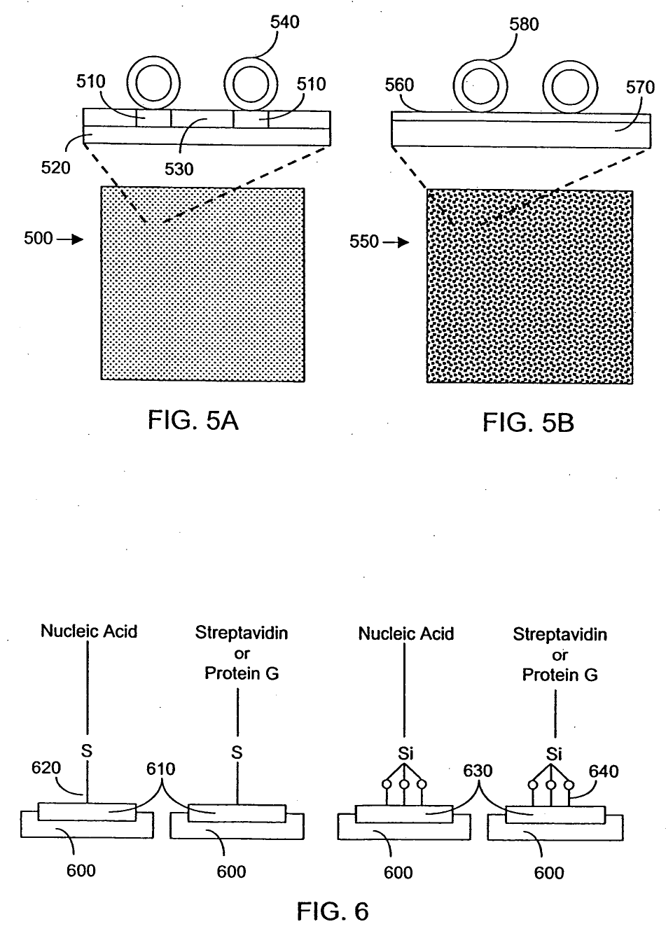

The invention arrays in one or more embodiments may include a substrate having two or more fixed locations with surface-attached coupling agents for binding to a reporter substrate that is conjugated to a probe. The substrate may be a rigid or flexible open platform or the entire array or chip may be enclosed within a housing. Since the probe molecule is identified by its reporter substrate rather than by its immobilization at a physical location on the array or chip surface, the array of fixed locations may be either regularly arranged (FIG. 5A) or randomly arranged (FIG. 5B) on the substrate. For example, a chip may be as small as 1 cm² and a subarray on such a chip containing about 1×10⁴ fixed locations may be as small as 1 mm×1 mm. The invention arrays provide the advantage that the array is reusable and procedures for its use may be varied according to the preferences of the user.

For example, as illustrated in FIG. 5A, in certain embodiments array 500 is made up of regularly spaced (for example 1 micron from center to center) adhere pads 510, which form fixed locations on substrate 520, with a protection layer 530 of chemically inert or insulator substance separating the adhere pads 510. Typically, the adhere pads are formed of an inorganic material such as gold, silica, plastic, aluminum oxide, platinum, and the like, and range in size from about 1 micron to less than 10 microns in largest dimension.

The adhere surfaces as illustrated in FIG. 6 (e.g., gold or glass) overlying the substrate are made adherent by surface modification with one or more surface-attached coupling agents, which are selected to form a specific binding pair with probe molecules or attachment sites in the set of probe-conjugated reporter substrates selected for use with the particular arrays. For example, as shown in detail in FIG. 2, the probe-conjugated reporter substrate 200 attaches via formation of specific binding pairs with surface attachment coupling agents 240, 460 on adhere pads 250 and reporter substrate 400 upon random contact. As shown in FIG. 5A, in a regular array the probe-conjugated reporter substrate 540 attaches to adhere pads 510, but does not attach to the protection layer 530 between the adhere pads 510. The regularly spaced adhere pads result in formation of a regular array of fixed location to which probe-conjugated reporter substrates 540 may be immobilized. Alternatively, as illustrated in FIG. 5B, in certain embodiments array 550 has a non-regular arrangement and includes an adhere surface 560 overlying substrate 570. In this case, the adhere surface may be formed of metal, glass or plastic with surface attachment coupling agents placed in a layer over the adhere surface 560. Probe-conjugated reporter substrates 580 bearing surface attached coupling agents that form a specific binding pair with those on the adhere surface 560 will randomly attach to the adhere surface to form a non-regular array of probe-conjugated reporter substrates.

Chip Surface Modification

The surface-attached coupling agents, in general, allow for attachment of the probe-conjugated reporter substrates covalently (for example, by crosslinking), non-covalently (for example, by binding or hybridization), or by self-assembly of the specific binding pair (for example, when poly(T) or streptavidin molecules are used). Techniques for formation of adhere surfaces or adhere pads at fixed locations on the array or chip by modification with surface-attached coupling agents will now be described with reference to FIGS. 6 through 9. In FIG. 6, substrate surface 600, with gold pad 610 formed thereon is modified by a compound 620 having a free thiol group that may form a specific binding pair with an oligonucleotide, streptavidin or Protein G. For example, a self-assembled monolayer (SAM) of organic compounds can be formed using a variety of commercially available thiol-containing molecules for attachment to a gold surface (Dojindo Corp., Gaithersberg, Md.).

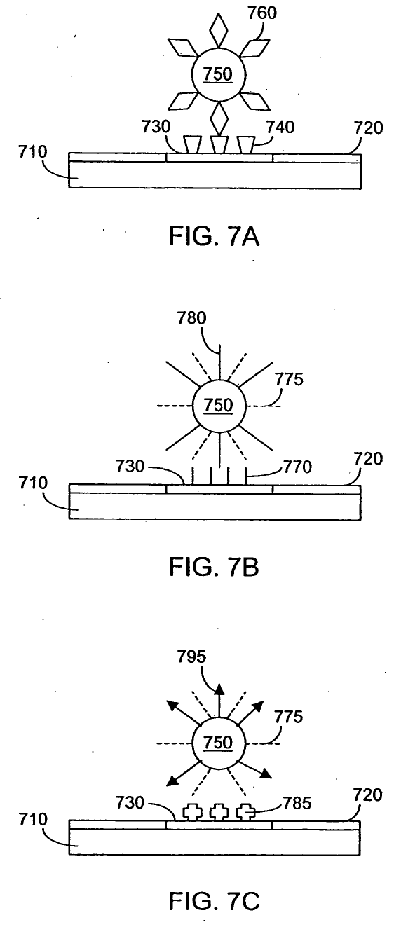

Further, substrate surface 600 with glass or silica pad 630 formed thereon is modified by a compound 640 having a free silane group 640 that may form a specific binding pair with an oligonucleotide, streptavidin or Protein G. As shown schematically in FIGS. 7A-C, the surface-attached coupling agent on the array is selected to form a specific binding pair with a coupling agent available on the surface of reporter substrates to be used in an assay. In FIG. 7A, substrate 710 is overlain with protection layer 720 and adhere pads 730, which are modified with surface-attached coupling agents Protein A or Protein G 740 to immobilize a probe-conjugated reporter substrate 750 decorated with antibody probes 760. As illustrated in FIG. 7B, by contrast, adhere pads 730 are modified with surface-attached coupling agents poly(T) 770 to immobilize a probe-conjugated reporter substrate 750 with nucleic acid probes 775 and decorated with poly(dA) coupling agent 780. As illustrated in FIG. 7C, adhere pads 730 are modified with surface-attached coupling agents streptavidin 785 to immobilize a probe-conjugated reporter substrate 750 with nucleic acid probes 775 and decorated with avidin coupling agents 795.

As illustrated in FIGS. 8A-B, the invention arrays may be configured in various formats. FIG. 8A illustrates a chip 800 with a flat substrate upon which probe-conjugated reporter substrates are immobilized in columns forming subarrays. Within a subarray, several probe-conjugated reporter substrates 810 are immobilized at a single adhere surface 820, illustrated in blow-up. As illustrated in FIG. 8B, chip 850 has three columnar subarrays in fluid channels 860, 861, 862 with probe-conjugated reporter substrates 870 randomly attached within the fluid channels. The density of the surface attached coupling agents on the array surface controls the density of the probe-conjugated reporter substrates that may be immobilized thereon.

Alternatively still, as shown in FIGS. 9A-B, the surface attached coupling agent on substrate 900 may be selected to form a binding pair with an organic molecule in a COIN label or COIN bead 910, 915, as described herein, leaving the probe molecules 920, 925 free for binding with an analyte in solution (for example a protein, polynucleotide, or chemical compound.

Thus, in addition to the oligonucleotide probes described herein with reference to sets of COIN-labeled probes for use in the invention systems and methods for sequencing polynucleotides, suitable probe molecules that can be incorporated into probe-conjugated reporter substrates for use with the invention arrays generally further include, without limitation, non-polymeric small molecules, antibodies, antigens, receptors, ligands, and the like.

Exemplary polypeptides suitable for use as a probes, for example, in making of probe-conjugated reporter substrates, as described herein, include, without limitation, a receptor for a cell surface molecule or fragment thereof; a lipid A receptor; an antibody or fragment thereof; peptide monobodies of the type a lipopolysacchardide-binding polypeptide; a peptidoglycan-binding polypeptide; a carbohydrate-binding polypeptide; a phosphate-binding polypeptide; a nucleic acid-binding polypeptide; and polypeptides that specifically bind to a protein-containing analyte. In certain examples, a set of probes may be antibodies specific for a set of particular protein-containing analytes or a particular class or family of protein-containing analytes.

A number of additional strategies aside from the inventive concept illustrated in FIG. 1 may be available for immobilizing the COIN-labeled probes and probe-conjugated reporter substrates used in the invention methods to the surface of an array, depending upon the type of surface attached coupling agent present on adhere surfaces of the array. For example, when the label is a COIN label, organic molecules on the surface of the COIN may provide or be provided with a specific binding partner for the surface attached coupling agent on the adhere surface of the array. When the label is provided by two or more COINs embedded within a polymeric microsphere, the polymeric exterior of the microsphere provides or is functionalized (see FIG. 2) to provide a specific binding partner for a coupling agent attached to the adhere surface of an array to form a fixed location. These strategies are also used in forming multiple arrays or subarrays on a chip surface according to the invention.

Thus, the available strategies for attaching the one or more probes or probe sets to adhere surfaces include, without limitation, covalently or non-covalently bonding (for example, in solution) one or more surface modified reporter substrates, COIN labels or COIN beads in the probe sets to adhere surface(s) on the surface of the array or chip. Such association may also include covalently or noncovalently attaching the COIN label or the microsphere to another moiety (a coupling agent), which in turn is covalently or non-covalently attached to the surface of the array structure via a surface attached coupling agent thereon.

Basically, adhere surface(s) of the array may be first modified (for example, primed) with a surface attached coupling agent which is attached to the surface thereof. This is achieved by providing a coupling agent precursor and then covalently or non-covalently binding the coupling agent precursor to the surface of the array (for example, at the fixed locations thereon). Once the adhere surface(s) of the array have been functionalized, the probe-conjugated Raman active label is exposed to the functional group attached to the array surface under conditions effective to (i) covalently or non-covalently bind to the coupling agent or (ii) displace the coupling agent such that the probe set covalently or non-covalently binds directly to the fixed locations making up the array. The binding of the probe-conjugated reporter substrate or COIN-labeled probes to the array is carried out under conditions that may be effective to allow the one or more functional groups thereon to remain available for binding to a specific binding pair on the COIN label or the COIN bead.

Suitable surface attached coupling agent precursors such as those used in FIG. 6 include, without limitation, silanes functionalized with an epoxide group, a thiol, or an alkenyl; and halide containing compounds. Silanes include a first moiety that binds to the surface of the array and a second moiety that binds to the COIN-labeled probe. Preferred silanes include, without limitation, 3-glycidoxypropyltrialkoxy-silanes with C1-6 alkoxy groups, trialkoxy(oxiranylalkyl)silanes with C2-12 alkyl groups and C1-6 alkoxy groups, 2-(1,2-epoxycyclohexyl)ethyltrialkoxysilane with C1-6 alkoxy groups, 3-butenyl trialkoxysilanes with C1-6 alkoxy groups, alkenyltrialkoxysilanes with C2-12 alkenyl groups and C1-6 alkoxy groups, tris[(1-methylethenyl)oxy]3-oxiranylalkyl silanes with C2-12 alkyl groups, [5-(3,3-dimethyloxiranyl)-3-methyl-2-pentenyl]trialkoxysilane with C1-6 alkoxy groups, (2,3-oxiranediyldi-2,1-ethanediyl)b-is-triethoxysilane, trialkoxy[2-(3-methyloxiranyl)alkyl]silane with C1-6 alkoxy groups and C2-12 alkyl groups, trimethoxy[2-[3-(17,17,17-trifluoro-heptadecyl)oxiranyl]ethyl]silane, tributoxy[3-[3-(chloromethyl)oxiranyl]-2-methylpropyl]silane, and combinations thereof. Silanes may be coupled to the array according to a silanization reaction scheme for which the conditions may be well known to those of skill in the art.

Thereafter, a probe set as described herein may be immobilized at adhere surfaces of an array according to the type of functionality provided by the coupling agent (see for example FIG. 6). Typically, a probe set may be attached to the coupling agent or displace the coupling agent for attachment to the array in aqueous conditions or aqueous/alcohol conditions. For example, epoxide functional groups may be opened to allow binding of amino groups, thiols or alcohols; and alkenyl functional groups may be reacted to allow binding of alkenyl groups.

The functional groups on the target analytes may also interact and bind to the modified adhere surface of the array. To preclude this from occurring, the substrate surface between the fixed locations defined by adhere surfaces of the array may also be provided with a protection layer by exposure to a blocking agent to minimize the number of sites where the analytes may attach to the surface of the array. The blocking agents may be structurally similar to the analytes, or may include such blocking agents as ethylene glycols or carbohydrates.

The term “chip” as used herein means a super structure comprising multiple arrays or subarrays, for example as depicted in FIG. 3 and FIG. 8. For example, a chip may be a substrate or surface containing multiple arrays. The arrays on the chip may be fluidically isolated by physical barrier structures, or the arrays may be in fluid communication to receive the same sample simultaneously or in sequence. The chip and/or the arrays thereon may be in any convenient shape, such as in square, strip and fluid or microfluid channel formats.

Still another embodiment of the invention is described now with reference to FIGS. 9A-B. In this embodiment, the invention provides methods for assaying a biological sample comprising at least one biomolecule using an invention array. The analyte biomolecules in the sample may be prelabeled by contact with a set of distinguishable optically active reporter molecules that bind specifically to different known biological analytes, wherein a member of the set binds specifically to a different known biomolecule and produces a distinguishable Raman-active signature associated with the biomolecule to which the member binds. Alternatively, in certain embodiments, biomolecules in the sample may be prelabeled with a reporter molecule that attaches to certain families of biomolecule, or indiscriminately to any protein, any polynucleotide, and the like.

As illustrated in FIG. 9A, the invention provides a one-step detection method based on use of the invention arrays wherein a detection complex 900 is formed on invention array 910. The detection complex is formed by contacting an invention array 910 with probe-conjugated reporter substrates, which include, respectively, reporter substrates 912, 915 and produce distinguishable Raman signatures, and further include biological probe molecules 922, 925, which bind specifically with different known biomolecules. Probe-conjugated reporter substrates 912, 915 bear surface attached coupling agents (not shown) that form a specific binding pair with those on the adhere surface of array 910.

A biological sample being tested for the presence of one or more known biomolecules is contacted with the array 910 and probe-conjugated reporter substrates 912, 915 under conditions suitable to promote formation of detection complex 900 in which a known biomolecule analyte may be captured, as shown by the probe 922. (The probe-conjugated reporter substrates may be immobilized on the array surface before or after contacting the sample, that is, before or after the probes conjugated to the reporter substrate capture a specific binding partner biomolecule).

In the one-step method, biomolecule analyte 930 (or the whole sample) is prelabeled with an optically active reporter molecule 940, which produces a signal (for example, fluorescence) distinguishable from the Raman signal of the reporter substrate. Formation of detection complex 900 is indicated by simultaneous detection of optical signals produced by the reporter substrate 910 and optically active reporter molecule 940 emanating from a fixed location on the array. By association of the optically active reporter molecule 940 with its known binding partner, biomolecule 930, the presence in the sample as well as the location on the array of the biomolecule 930 is determined. By contrast, detection of an optical signal from reporter substrate 915 unaccompanied by the presence of second optical signal from a reporter molecule, such as 940, indicates a negative result for the biomolecule to which probe 925 binds specifically. The one-step method is particularly suitable for drug screening, in which, for example, drug target candidates may be attached to a first probe set and immobilized on a surface, and the drug candidates may be attached to a second probe set. In this manner, drug and drug target may be identified efficiently.

The invention methods may also be performed as a two-step sandwich-type assay as illustrated in FIG. 9B in which the binding complex formed by capture of the biomolecule analyte 930 is contacted with a second probe conjugate comprising a second probe molecule 950. The second probe molecule may be or include an antibody that binds specifically a known biomolecule 930, and a distinguishable optically active reporter molecule 960. If the second probe molecule binds specifically to a known biomolecule 960, optically active reporter molecule 960 may produce an optical signal that is associated with the known biomolecule to which probe 950 binds specifically. In certain embodiments, a set of probe conjugates are used to contact the binding complexes so formed, wherein members of the set of probe conjugates collectively bind specifically to different known biomolecules and produce distinguishable Raman-active signatures that are individually associated with the particular biomolecule to which the member binds. COIN labels may be used as either one or both of the reporter substrate and the label for the second probes in these assay methods.

The analytes that can be detected using the invention methods include drugs, metabolites, pesticides, pollutants, and the like. Included among drugs of interest are the alkaloids. Among the alkaloids are morphine alkaloids, which includes morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites.

The term analyte further includes polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, restriction enzymes, activators, repressors, nucleases, polymerases, histones, repair enzymes, chemotherapeutic agents, and the like.

The analyte may be a molecule found directly in a sample such as a body fluid from a host. The sample may be examined directly or may be pretreated to render the analyte more readily detectible. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid may be, for example, urine, blood, plasma, serum, saliva, semen, stool, sputum, cerebral spinal fluid, tears, mucus, and the like.

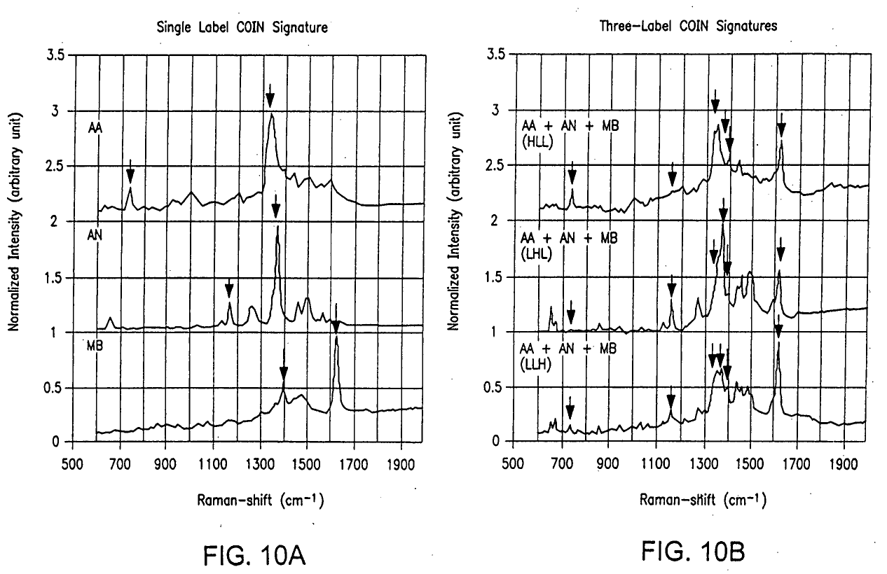

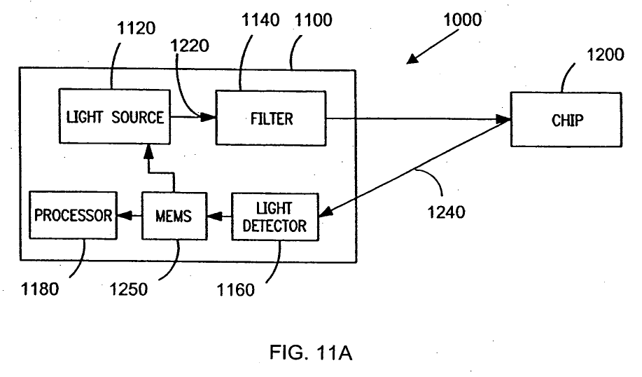

FIGS. 10A and 10-B are graphs showing SERS signatures of COINs made with individual (FIG. 10A) or mixtures (FIG. 10B) of Raman labels. FIGS. 10A and 10B show COIN signatures in multiplex detection. COINs were made with individual or mixtures of Raman labels at concentrations from 2.5 μM to 20 μM, depending on signatures desired: 8-aza-adenine (AA), 9-aminoacridine (AN), methylene blue (MB). Representative peaks are indicated by arrows; peak intensity values have been normalized to respective maximums; the Y axis values are in arbitrary unit; spectra are offset by 1 unit from each other. FIG. 10A shows signatures of COINs made with the three Raman labels, respectively, showing that each label produced a unique signature. FIG. 10B shows signatures of COINs made from mixtures of the 3 Raman labels at concentrations that produced signatures as indicated: HLL means high peak intensity for AA (H) and low peak intensity for both AN (L) and MB (L); LHL means low peak intensity for AA (L), high peak intensity for AN (H) and Low for MB (L); LLH means low for both AA (L) and AN (L) and high for MB (H). Note that peak heights could be adjusted by varying label concentrations, but they might not necessarily be proportional to label concentrations used due to different adsorption affinity of the Raman labels on metal surfaces. See also Table 1 for further examples.

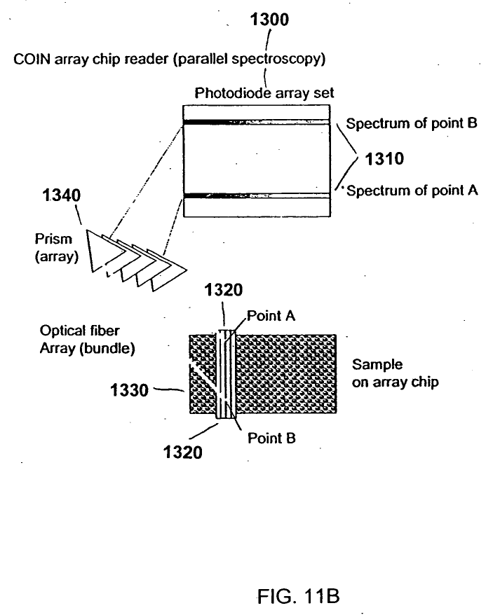

An apparatus used in performing the invention methods will now be described with reference to FIG. 11A. In apparatus 1000 Raman analyzer 1100 emits a beam of light 1220 from a light source 1120, to the surface of chip 1200, from which it is reflected back as scattered beam 1240. Spectroscope light detector 1160 receives scattered beam 1240, filtered through MEMS device 1250 and provides a signal representative of a spectrum of the scattered light to processor 1180. Raman analyzer 1100 may further include filter or prism 1140 to select a predetermined bandwidth of beam of light 1220 directed to chip 1200. On chip 1200, binding of a target biomolecule to a probe molecule, for example in a detection complex, causes a frequency shift in the spectrum of the scattered light beam 1240 detected by spectroscope light detector 1160 corresponding to a defined location on chip 1200, which detection is passed on to processor 1180. Two or more spectroscopes operating in parallel may be used for multiplex detection of signals from two or more locations on a chip surface (see FIG. 11B for example). As discussed herein, multiple subarrays on a chip can be scanned in a high throughput manner to effect rapid assay of, for example, the sequence of a polynucleotide, or to determine the presence of various biomolecules in a complex biological sample. FIG. 11B is an illustrative COIN array chip reader used in one aspect of the invention for detecting multiple signals. Such a reader includes parallel photodiode array sets 1300 to collect multiple spectra 1310 simultaneously from a sample 1320 on an array chip 1330 and may be used with an apparatus of FIG. 11A. As described above in FIG. 11A, the Raman analyzer 1100 may further include filter or prism 1140 (also shown as 1340 in FIG. 11B) to select a predetermined bandwidth of beam of light 1220 directed to chip 1200.

In certain embodiments of the invention, the metal particles used in COIN labels and other reporter substrates, as described herein, may be formed from metal colloids. As used herein, the term “colloid” refers to a category of complex fluids consisting of nanometer-sized particles suspended in a liquid, usually an aqueous solution. During metal colloid formation or “growth” in the presence of organic molecules in the liquid, the organic molecules may be adsorbed on the primary metal crystal particles suspended in the liquid and/or in interstices between primary metal crystal particles. Typical metals contemplated for use in formation of nanoparticles from metal colloids include, for example, silver, gold, platinum, copper, aluminum, and the like. A typical average size range for the metal particles in the colloids used in manufacture of the nanoparticles used in the invention methods and compositions are from about 8 nm to about 15 nm. These metal colloids may be used to provide metal “seed” particles that may be used to generate enlarged metal particles, or aggregates, having an average size range from about 20 nm to about 30 nm.

As used herein, the term “organic compound” refers to any hydrocarbon molecule containing at least one aromatic ring and at least one nitrogen atom. “Organic compounds” may also contain atoms such as O, S, P, and the like. As used herein, “Raman-active organic compound” refers to an organic molecule that produces a unique SERS signature in response to excitation by a laser. A variety of organic compounds, both Raman-active and non-Raman active, may be contemplated for use as components in nanoparticles. In certain embodiments, Raman-active organic compounds may be polycyclic aromatic or heteroaromatic compounds. Typically the Raman-active compound has a molecular weight less than about 500 Daltons.

In addition, it is understood that these Raman-active compounds may include fluorescent compounds or non-fluorescent compounds. Exemplary Raman-active organic compounds include, but may be not limited to, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, 9-amino-acridine, and the like.

Additional, non-limiting examples of Raman-active organic compounds include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like. These and other Raman-active organic compounds may be obtained from commercial sources (for example, Molecular Probes, Eugene, Ore.). Chemical structures of exemplary Raman-active organic compounds are shown in Table 1 below.

In certain embodiments, the Raman-active compound is adenine, 4-amino-pyrazolo(3,4-d)pyrimidine, or 2-fluoroadenine. In one embodiment, the Raman-active compound is adenine.

When fluorescent compounds are incorporated into nanoparticles described herein, the compounds include, but are not limited to, dyes, intrinsically fluorescent proteins, lanthanide phosphors, and the like. Dyes include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me2, N-coumarin-4-acetate, 7-OH-4-CH3-coumarin-3-acetate, 7-NH2-4CH3-coumarin-3-acetate (AMCA), monobromobimane, pyrene trisulfonates, such as Cascade Blue, and monobromotrimethyl-ammoniobimane.

As used herein the term “distinguishable” as applied to a Raman or fluorescent signal or signature, means that individual probes in a set of probes with different binding specificities used in an assay are labeled with reporter substrates, such as fluorescent molecules, or COIN labels that produce a one or more optical signals that can be separately detected. For Raman signatures, detection of the “distinguishable” Raman signal and a knowledge of the target molecule of the attached probe is sufficient to identify the presence of the analyte target of the probe in the sample being assayed, whether the analyte-probe-COIN complex is attached to a solid surface or in solution. Unique Raman signatures may be created within a set of COIN labeled probes used in the invention methods by using different Raman labels, different mixtures of Raman labels and different ratios of Raman labels for labeling individual probes within a set of probes. High sensitivity of the invention assay methods is achieved by incorporating many, indeed up to thousands, of Raman-active molecules in a single COIN label. FIGS. 10A-B are graphs showing SERS signatures of COINs made with individual (FIG. 11A) or mixtures (FIG. 11B) of three Raman labels. Referring to FIGS. 10A and 10B, graphs are shown illustrating SERS signatures of COINs made with individual (FIG. 10A) or mixtures (FIG. 10B) of Raman labels 8-aza-adenine (AA), 9 aminoacridine (AN), and methylene blue (MB). HLL=relatively high peak intensity for AA (H) and relatively low peak intensity for both AN (L) and MB (L): LHL=relatively low, high and low peak intensity for AA (L), AN (H) and MB (L), respectively; LLH=relatively low for both AA (L) and AN (L) and high for MB (H).

TABLE 1

| No | Name | Structure |
|---|---|---|
| 1 | 8-Aza-Adenine | 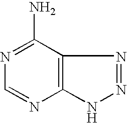 |
| 2 | N-Benzoyladenine | 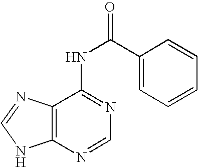 |
| 3 | 2-Mercapto-benzimidazole (MBI) | 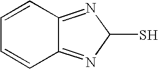 |
| 4 | 4-Amino-pyrazolo[3,4-d]pyrimidine | 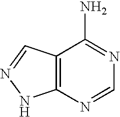 |
| 5 | Zeatin | 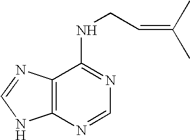 |
| 6 | Methylene Blue | 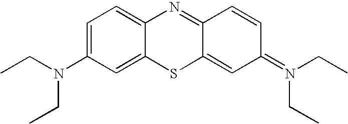 |
| 7 | 9-Amino-acridine | 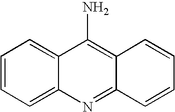 |
| 8 | Ethidium Bromide | 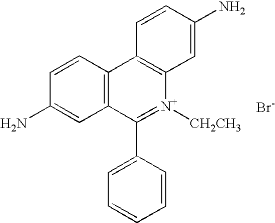 |
| 9 | Bismarck Brown Y | 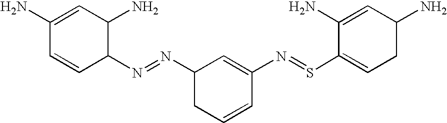 |
| 10 | 1. N-Benzyl-aminopurine | 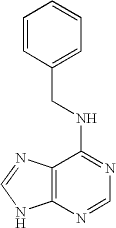 |
| 11 | Thionin acetate | 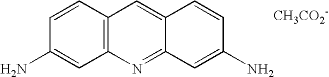 |
| 12 | 3,6-Diaminoacridine | 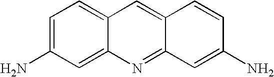 |
| 13 | 6-Cyanopunne | 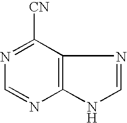 |
| 14 | 4-Amino-5-imidazole-carboxamide hydrochloride | 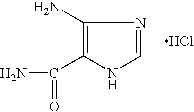 |
| 15 | 1,3-Diiminoisoindoline | 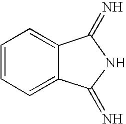 |
| 16 | Rhodamine 6G | 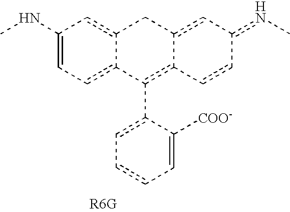 |
| 17 | Crystal Violet | 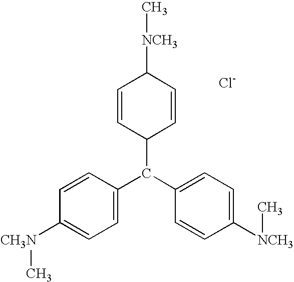 |
| 18 | Basic Fuchsin | 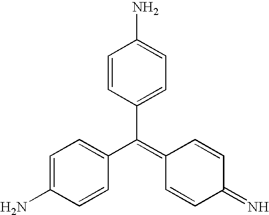 |
| 19 | Aniline Blue diammonium salt | 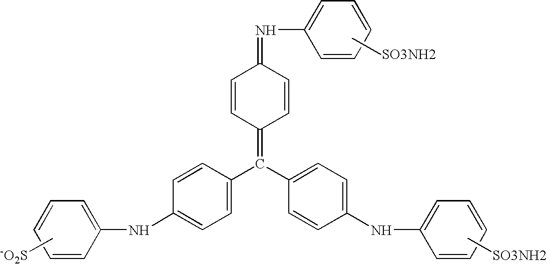 |
| 20 | N-[(3-(Anilinomethylene)-2-chloro-1- cyclohexen-1-yl)methylene]aniline monohydrochloride | 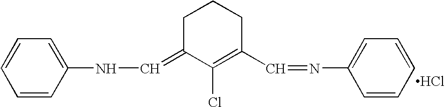 |
| 21 | O-(7-Azabenzotriazol-1-yl)-N,N,N′,N′- tetramethyluronium hexafluorophosphate | 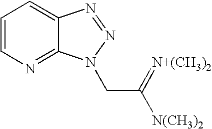 |
| 22 | 9-Aminofluorene hydrochloride | 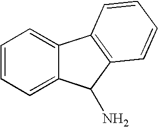 |
| 23 | Basic Blue | 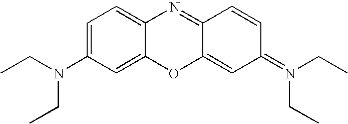 |
| 24 | 1,8-Diamino-4,5-dihydroxyanthraquinone | 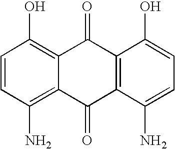 |
| 25 | Proflavine hemisulfate salt hydrate | 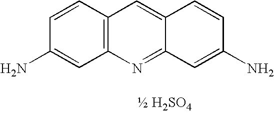 |
| 26 | 2-Amino-1,1,3-propenetricarbonitrile | 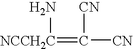 |
| 27 | Vanamine Blue RT Salt | 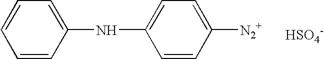 |
| 28 | 4,5,6-Triaminopyrimidine sulfate salt | 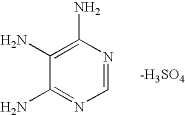 |
| 29 | 2-Amino-benzothiazole | 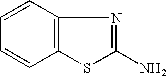 |
| 30 | Melamine | 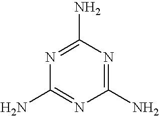 |
| 31 | 3-(3-Pyridylmethylamino)propionitrile | 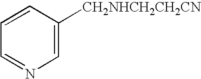 |
| 32 | Silver(I) sulfadiazine | 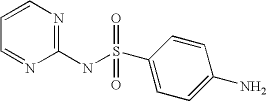 |
| 33 | Acrifiavine | 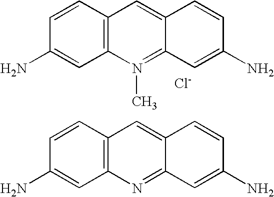 |
| 34 | 4-Amino6-Mercaptopyrazolo[3,4- d]pyrimidine | 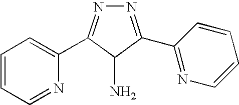 |
| 35 | 2-Am-Purine | 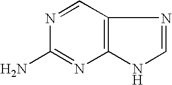 |
| 36 | Adenine Thiol | 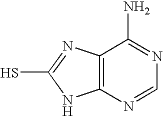 |
| 37 | F-Adenine | 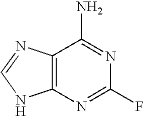 |
| 38 | 6-Mercaptopurine | 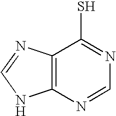 |
| 39 | 4-Amino-6-mercaptopyrazolo[3,4-d]pyrimidine | 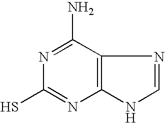 |
| 40 | Rhodamine 110 | 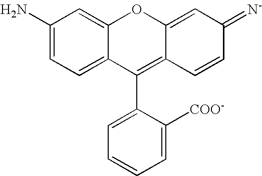 |

The COIN particles may be readily prepared using standard metal colloid chemistry. COIN, comprising an aggregation of metal seed particles, may be 50 to 200 nm in average diameter and multiple COIN, for example as many as about 100 COIN, may be embedded in a polymer bead that has an average diameter in the range from about 1 micron to about 10 microns to form a COIN bead.

COIN particles may be formed by particle growth in the presence of organic compounds. The preparation of such nanoparticles also takes advantage of the ability of metals to adsorb organic compounds. Indeed, since Raman-active organic compounds adsorb onto the metal during formation of the metallic colloids, many Raman-active organic compounds may be incorporated into a nanoparticle without requiring special attachment chemistry.

In certain embodiments, primary COINs (for example, less than 60 nm) may be aggregated to form stable clustered structures, which range in size from about 35 nm to about 200 nm, for example about 50 nm to about 200 nm.

The nanoparticles according to the invention may be prepared by a physico-chemical process called Organic Compound Assisted-Metal Fusion (OCAMF), also sometimes referred to as organic compound-induced Particle Aggregation and Coalescence (PAC). In SERS, the enhancement may be attributed primarily to an increase in the electromagnetic field on curved surfaces of coinage metals. It is also known that chemical enhancement (CE) may be obtained by placing molecules in a close proximity to metal surfaces. Theoretical analysis predicts that electromagnetic enhancement (EME) is particularly strong on rough edges of metal particles.

These composite organic-inorganic nanoparticles (COIN) may be used as label or reporter or as reporter substrate when conjugated to various types of probes used in the invention both for proteinaceous molecules and for nucleotide sequences. According to the COIN concept, the interaction between the organic Raman label molecules and the metal colloids has mutual benefits. Besides serving as signal sources, the organic molecules promote and stabilize metal particle association that is in favor of EME of SERS. On the other hand, the metal crystal structures provide spaces to hold and stabilize Raman label molecules, especially those in the junction between primary metal crystal particles in a cluster of such particles.

In general, COINs may be prepared as follows. An aqueous solution is prepared containing suitable metal cations, a reducing agent, and at least one suitable Raman-active organic compound. The components of the solution may be then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This type of nanoparticle is a cluster of several primary metal crystal particles with the Raman-active organic compound trapped in the junctions of the primary particles or embedded in the metal crystals.

In another aspect, the COINs may include a second metal different from the first metal, wherein the second metal forms a layer overlying the surface of the COIN. To prepare this type of nanoparticle, COINs may be placed in an aqueous solution containing suitable second metal cations and a reducing agent. The components of the solution may be then subjected to conditions that reduce the second metallic cations, thereby forming a metallic layer overlying the surface of the nanoparticle. In certain embodiments, the second metal layer includes metals, such as, for example, silver, gold, platinum, aluminum, copper, zinc, iron, and the like. COINs range in size from about 50 nm to 200 nm.

In certain embodiments, the metallic layer overlying the surface of the nanoparticle is referred to as a protection layer. This protection layer contributes to aqueous stability of the colloidal nanoparticles. As an alternative to a metallic protection layer, or in addition to metallic protection layers, COINs may be coated with a layer of silica. If the COINs have already been coated with a metallic layer, for example, gold, a silica layer may be attached to the gold layer by vitreophilization of the COINs with, for example, 3-aminopropyltrimethoxysilane (APTMS). Silica deposition is initiated from a supersaturated silica solution, followed by growth of a silica layer by dropwise addition of ammonia and tetraethyl orthosilicate (TEOS). The silica-coated COINs may be readily functionalized using standard silica chemistry. In alternative embodiments, titanium oxide or hematite may be used as a protection layer.

In certain other embodiments, COINs may include an organic layer overlying the metal layer or the silica layer. Typically, these types of nanoparticles may be prepared by covalently attaching organic compounds to the surface of the metal layer of COINs. Covalent attachment of an organic layer to the metallic layer may be achieved in a variety ways well known to those skilled in the art, for example, through thiol-metal bonds. In alternative embodiments, the organic molecules attached to the metal layer may be crosslinked to form a solid molecular network coating. An organic layer may also be used to provide colloidal stability and functional groups for further derivatization of the COIN.

An exemplary organic layer is produced by adsorption of an octylamine modified polyacrylic acid onto COINs, the adsorption being facilitated by the positively charged amine groups. The carboxylic groups of the polymer may be then crosslinked with a suitable agent such as lysine, (1,6)-diaminoheptane, and the like. Unreacted carboxylic groups may be used for further derivation. Other functional groups may be also introduced through the modified polyacrylic backbones. The functional groups may be used for attachment of the COIN to the surface of a substrate and to attach probes to the COIN.

Attachment of a probe to or inclusion of a probe in the organic layer via specific binding partners is especially useful in the detection of biological molecules, which may be referred to herein as “biomolecules”. In certain embodiments, exemplary probes may be antibodies, antigens, polynucleotides, oligonucleotides, receptors, ligands, and the like. In other embodiments, the organic layer may include or have attached thereto via specific binding partners a polynucleotide probe.

The probes attached to or incorporated into organic surface molecules of the COIN in certain embodiments may be selected to bind specifically to molecular epitopes, for example, receptors, lipids, peptides, cell adhesion molecules, polysaccharides, biopolymers, and the like, presented on the surface membranes of cells or within the extracellular matrix of biomolecular analytes or to oligonucleotide sequences. A wide variety of probes, including but not limited to antibodies, antibody fragments, peptides, small molecules, polysaccharides, nucleic acids, aptamers, peptidomimetics, and oligonucleotides, alone or in combination, may be utilized to specifically bind to cellular epitopes and receptors contained in analytes of interest in biological samples. These probes may be attached to a COIN surface or derivatized COIN surface covalently (direct-conjugation) or noncovalently (indirect conjugation).

For example, avidin or streptavidin-biotin specific binding partners may be extremely useful noncovalent systems that have been incorporated into many biological and analytical systems. Avidin has a high affinity for biotin (10⁻¹⁵ M), facilitating rapid and stable binding under physiological conditions. Attachment of one or more probes to a single COIN, as described herein, may be accomplished utilizing this approach in two or three steps, depending on the formulation, to complete the COIN-avidin-probe “sandwich”. In fact, the COIN surface may be decorated with a multiplicity of probe molecules using this technique. Alternatively, avidin, with four, independent biotin binding sites provides the opportunity for attachment of multiple COIN having biotin surface molecules to an avidin-derivatized defined location (for example an “adhere surface”) on a substrate surface, as described herein.

As used herein, a “probe” may be any molecule that binds to another molecule and, as the term is used in this application, refers to a small targeting molecule that binds specifically to another molecule on a biological surface separate and distinct from the reporter substrate, such as a COIN, to which it is attached. The reaction does not require, nor exclude, a molecule that donates or accepts a pair of electrons to form a coordinate covalent bond with a metal atom of a coordination complex. Conjugations may be performed before or after an organic coating is applied to the COIN, depending upon the probe employed. Direct chemical conjugation of probes to proteinaceous molecules, for example in proteinaceous reporter substrates, often takes advantage of numerous amino-groups (for example, lysine) inherently present within the surface. Another common post-processing approach is to activate surface carboxylates with carbodiimide prior to probe addition. The selected covalent linking strategy is primarily determined by the chemical nature of the probe. Monoclonal antibodies and other large proteins may denature under harsh processing conditions; whereas, the bioactivity of carbohydrates, short peptides, nucleic acids, aptamers, or peptidomimetics often may be preserved. To ensure high probe binding integrity and maximize avidity for the organic molecule of the COIN, flexible polymer spacer arms, for example, polyethylene glycol, amino acids or simple caproate bridges, may be inserted between an activated surface functional group and the probe. These extensions may be 10 nm, or longer, and minimize interference of probe binding by COIN surface interactions.

Monoclonal Antibody and Fragments

Rapid expansion of the monoclonal antibody industry has provided a plethora of antibody probes that may be directed against a wide spectrum of pathologic molecular epitopes. Antibodies or their fragments may be from several classes including IgG, IgM, IgA, IgE or IgD. Immunoglobin-gamma. (IgG) class monoclonal antibodies have been most often conjugated to various surfaces to provide active, site-specific targeting. These proteins may be symmetric glycoproteins (MW ca. 150,000 daltons) composed of identical pairs of heavy and light chains. Hypervariable regions at the end of each of two arms provide identical antigen-binding domains. A variably sized branched carbohydrate domain is attached to complement-activating regions, and the hinge may contain particularly accessible interchain disulfide bonds that may be reduced to produce smaller fragments.

Bivalent F(ab′)₂ and monovalent F(ab) fragments may be derived from selective cleavage of the whole antibody by pepsin or papain digestion, respectively. Elimination of the Fc region greatly diminishes the size of the probe molecule.

Most monoclonal antibodies may be of murine origin and may be inherently immunogenic to varying extents in other species. Humanization of murine antibodies through genetic engineering or other combinatorial chemical methods have led to development of chimeric ligands with improved binding affinity.

Phage Display

Phage display techniques may be now used to produce recombinant (for example, human) monoclonal antibody fragments against a large range of different antigens without involving antibody-producing animals. In general, cloning creates large genetic libraries of corresponding DNA (CDNA) chains deducted and synthesized by means of the enzyme “reverse transcriptase” from total messenger RNA (mRNA) of B-lymphocytes. Immunoglobulin cDNA chains may be amplified by PCR (polymerase chain reaction) and light and heavy chains specific for a given antigen may be introduced into a phagemid vector. Transfection of this phagemid vector into the appropriate bacteria results in the expression of an scFv immunoglobulin molecule on the surface of the bacteriophage. Bacteriophages expressing specific immunoglobulin may be selected by repeated immunoadsorption/phage multiplication cycles against desired antigens (for example, proteins, peptides, nuclear acids, and sugars). Bacteriophages strictly specific to the target antigen may be introduced into an appropriate vector, (for example, Escherichia coli, yeast, cells) and amplified by fermentation to produce large amounts of antibody fragments with structures very similar to natural antibodies. (De Bruin et al., Selection of high-affinity phage antibodies from phage display libraries. Nat Biotechnol. 1999; 17:397-399; Stadler, Antibody production without animals. Dev Biol Stand. 1999; 101:45-48; Wittrup, Phage on display, Trends Biotechnol. 1999; 17:423-424; Sche et al., Display cloning: functional identification of natural product receptors using cDNA-phage display. Chem Biol. 1999; 6:(707-716).

Peptides

Peptides, like antibodies, may have high specificity and epitope affinity for use as COIN probes. These may be small peptides (5 to 10 amino acids) specific for a unique receptor sequences (for example, the RGD epitope of various molecules involved in inflammation or larger, biologically active hormones such as cholecystokinin). Peptides or peptide (nonpeptide) analogues of cell adhesion molecules, cytokines, selectins, cadhedrins, Ig superfamily, integrins and the like may be utilized for COIN probes.

Asialoglycoproteins and Polysaccharides

Asialoglycoproteins (ASG) have been used as probes for liver-specific diseases due to their high affinity for ASG receptors located uniquely on hepatocytes. ASG probes have been used to detect primary and secondary hepatic tumors as well as benign, diffuse liver disease such as hepatitis. The ASG receptor is highly abundant on hepatocytes, approximately 500,000 per cell, rapidly internalizes and is subsequently recycled to the cell surface. Polysaccharides such as arabinogalactan may also be utilized as probes for hepatic targets. Arabinogalactan has multiple terminal arabinose groups that display high affinity for ASG hepatic receptors.

Aptamers

Aptamers may be high affinity, high specificity RNA or DNA-based probes produced by in vitro selection experiments. Aptamers may be generated from random sequences of 20 to 30 nucleotides, selectively screened by absorption to molecular antigens or cells, and enriched to purify specific high affinity binding ligands. In solution, aptamers may be unstructured but may fold and enwrap target epitopes providing specific binding recognition. The unique folding of the nucleic acids around the epitope affords discriminatory intermolecular contacts through hydrogen bonding, electrostatic interaction, stacking, and shape complementarity. In comparison with protein-based ligands, aptamers may be stable and may be more conducive to heat sterilization. Aptamers may be currently used to target a number of clinically relevant pathologies including angiogenesis, activated platelets, and solid tumors and their use is increasing.

Polynucleotides

The term “polynucleotide” is used broadly herein to mean a sequence of deoxyribonucleotides or ribonucleotides that may be linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least 6 nucleotides to about 9 nucleotides in length. Polynucleotide probes used in the invention methods for sequencing a polynucleotide may be useful for detecting and hybridizing under suitable conditions to complementary polynucleotides in a biological sample and may be used in DNA sequencing by pairing a known polynucleotide probe with a known Raman-active COIN comprising one or more Raman-active organic compounds, as described herein. The nucleotides of a polynucleotide sequence may be generally ligated by a covalent phosphodiester bond. However, the covalent bond also may be any of numerous other bonds, including a thiodiester bond, a phosphorothioate bond, a peptide-like amide bond or any other bond known to those in the art as useful for linking nucleotides to produce synthetic polynucleotides. The incorporation of non-naturally occurring nucleotide analogs or bonds linking the nucleotides or analogs may be particularly useful where the polynucleotide is to be exposed to an environment that may contain a nucleolytic activity, including, for example, a tissue culture medium, since the modified polynucleotides may be less susceptible to degradation.

As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying the selected nucleotide sequence. It will be recognized that some amount of non-specific hybridization is unavoidable, but is acceptable provided that hybridization to a target nucleotide sequence is sufficiently selective such that it may be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (for example, homologous) nucleic acid molecule other than the target nucleic acid molecule. Conditions that allow for selective hybridization may be determined empirically, or may be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42EC (moderate stringency conditions); and 0.1×SSC at about 68EC (high stringency conditions). Washing may be carried out using only one of these conditions, for example, high stringency conditions, or each of the conditions may be used, for example, for 10-15 minutes each, in the order listed above, repeating any or all of the steps listed. However, as mentioned above, optimal conditions will vary, depending on the particular hybridization reaction involved, and may be determined empirically.

As used herein, the term “antibody” is used in its broadest sense to include polyclonal and monoclonal antibodies, as well as antigen binding fragments of such antibodies. An antibody useful as a capture probe in an invention array or chip, or an antigen-binding fragment thereof, is characterized, for example, by having specific binding activity for an epitope of an analyte. The antibody, for example, includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies may be constructed using solid phase peptide synthesis, may be produced recombinantly or may be obtained, for example, by screening combinatorial libraries consisting of variable heavy chains and variable light chains. These and other methods of making, for example, chimeric, humanized, CDR-grafted, single chain, and bifunctional antibodies may be well known to those skilled in the art.

The term “binds specifically” or “specific binding activity,” when used in reference to an antibody means that an interaction of the antibody and a particular epitope has a dissociation constant of at least about 1×10^{-6,} generally at least about 1×10^{-7,} usually at least about 1×10^{-8,} and particularly at least about 1×10⁻⁹ or 1×10⁻¹⁰ or less. As such, Fab, F(ab′)2, Fd and Fv fragments of an antibody that retain specific binding activity for an epitope of an antigen, may be included within the definition of an antibody.

In the context of the invention, the term “ligand” denotes a naturally occurring specific binding partner of a receptor, a synthetic specific-binding partner of a receptor, or an appropriate derivative of the natural or synthetic ligands. As one of skill in the art will recognize, a molecule (or macromolecular complex) may be both a receptor and a ligand. In general, the binding partner having a smaller molecular weight is referred to as the ligand and the binding partner having a greater molecular weight is referred to as a receptor. A probe may also be a ligand.

In its broadest terms, the invention provides methods for detecting an analyte in a sample. Such methods may be performed, for example, by contacting a sample containing an analyte with a reporter substrate including or conjugated to a probe, wherein the probe binds to the analyte; and detecting SERS signals emitted by the reporter substrate, wherein the signals may be indicative of the presence of a particular known analyte. More commonly, the sample contains a pool of biological analytes and the sample is contacted with a set of COIN-labeled probes, as described herein, wherein a member of the set is provided with a probe that binds specifically to a known biological analyte (for example, a polynucleotide) and a different combination of Raman-active organic compounds may be incorporated into members of the set to provide a distinguishable Raman signature unique to the set so the Raman signature may readily be correlated with the known analyte to which the probe will bind specifically.

In the invention methods for sequencing a polynucleotide, the organic layer in the COIN has an attached nucleotide sequence, for example, a DNA sequence, as probe and the “analyte” or “target” of the probe is a complementary nucleotide sequence. In other aspects of the invention methods and devices, the analyte may be included of a member of a specific binding pair (sbp) and may be a ligand, which is monovalent (monoepitopic) or polyvalent (polyepitopic), usually antigenic or haptenic, and is a single compound or plurality of compounds which share at least one common epitopic or determinant site. The analyte may be a part of a cell such as bacteria or a cell bearing a blood group antigen such as A, B, D, etc., or an HLA antigen or a microorganism, for example, a bacterium, fungus, protozoan, or virus.

A member of a specific binding pair (“sbp member”) is one of two different molecules, having an may be a on the surface or in a cavity which specifically binds to and is thereby defined as complementary with a particular spatial and polar organization of the other molecule. The members of the specific binding pair may be referred to as ligand and receptor (antiligand) or analyte and probe. These will usually be members of an immunological pair such as antigen-antibody, although other specific binding pairs such as biotin-avidin, hormones-hormone receptors, nucleic acid duplexes, Immunoglobulin G-protein A, polynucleotide pairs such as DNA-DNA, DNA-RNA, and the like may be not immunological pairs, but may be included in the definition of sbp member.

Specific binding is the specific recognition of one of two different molecules for the other compared to substantially lesser recognition of other molecules. Generally, the molecules have may be as on their surfaces or in cavities giving rise to specific recognition between the two molecules. Exemplary of specific binding may be antibody-antigen interactions, enzyme--substrate interactions, polynucleotide hybridization interactions, and so forth.

Non-specific binding is non-covalent binding between molecules that is relatively independent of specific surface structures. Non-specific binding may result from several factors including hydrophobic interactions between molecules.

The invention methods, systems and apparatus may be used to detect the presence of a particular target analyte, for example, a nucleic acid, polynucleotide, protein, enzyme, antibody or antigen or to screen bioactive agents, i.e. drug candidates, for binding to a particular target or to detect the presence of agents, such as pollutants in a soil, water or gas sample. As discussed above, any analyte for which a probe moiety, such as a peptide, protein, oligonucleotide or aptamer, may be designed may be used in combination with the disclosed COIN labels and other reporter substrates.

The monoepitopic ligand analytes will generally be from about 100 to 2,000 molecular weight, more usually from 125 to 1,000 molecular weight. The analytes include drugs, metabolites, pesticides, pollutants, and the like. Included among drugs of interest may be the alkaloids. Among the alkaloids may be morphine alkaloids, which includes morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites.

The term analyte further includes polynucleotide analytes such as those polynucleotides defined below. These include m-RNA, r-RNA, t-RNA, DNA, DNA-RNA duplexes, etc. The term analyte also includes receptors that may be polynucleotide binding agents, such as peptide nucleic acids (PNA), restriction enzymes, activators, repressors, nucleases, polymerases, histones, repair enzymes, chemotherapeutic agents, and the like.

The analyte may be a molecule found directly in a sample, such as a body fluid from a host or patient. The sample may be examined directly or may be pretreated to render the analyte more readily detectible. Furthermore, the analyte of interest may be determined by detecting an agent probative of the analyte of interest, such as a specific binding pair member complementary to the analyte of interest, whose presence will be detected only when the analyte of interest is present in a sample. Thus, the agent probative of the analyte becomes the analyte that is detected in an assay. The body fluid may be, for example, urine, blood, plasma, serum, saliva, semen, stool, sputum, cerebral spinal fluid, tears, mucus, and the like.

The following paragraphs include further details regarding exemplary methods of using COIN-labeled probes (composite organic-inorganic nanoparticles (COIN) having a probe molecule conjugated thereto) and other probe-conjugated reporter substrates in assay of biomolecules. It will be understood that numerous additional specific examples of applications that utilize COIN-labeled probes may be identified using the teachings of the present specification. One of skill in the art will recognize that many interactions between polypeptides and their specific binding target molecules may be detected using COIN-labeled polypeptides. In one group of exemplary applications, COIN labeled antibodies (antibodies conjugated to a COIN) may be used to detect interaction of the COIN labeled antibodies with antigens, either in solution or on a solid support (for example, immobilized on an array adhere surface). Such assays differ from conventional immunoassays in that the signal amplification step is unnecessary. In another example, a COIN labeled enzyme is used to detect interaction of the COIN-labeled enzyme with a substrate.

In the methods of the invention, a “sample” may include a wide variety of analytes that may be analyzed using the probe-conjugated reporter substrates described herein. For example, a sample may be an environmental sample, such as atmospheric air, ambient air, water, sludge, soil, and the like. In addition, a sample may be a biological sample, including, for example, a subject's breath, saliva, blood, urine, feces, various tissues, and the like.

Commercial applications for methods employing the COIN-labeled probes and probe-conjugated reporter substrates described herein include environmental toxicology and remediation, biomedicine, materials quality control, monitoring of food and agricultural products for the presence of pathogens, medical diagnostics, detection and classification of bacteria and microorganisms both in vitro and in vivo for biomedical uses and medical diagnostic uses, law enforcement applications (for example, DNA testing), food/beverage/agriculture applications, freshness detection, fruit ripening control, fermentation process monitoring and control applications, flavor composition and identification, product quality and identification, product quality testing, personal identification, product identity monitoring, biological weapons detection, infectious disease detection and breath applications, body fluids analysis, drug discovery, and the like.

A variety of analytical techniques may be used to analyze the Raman signatures of the constructs containing Raman-active organic compounds, such as the COIN particles described herein. Such techniques include for example, nuclear magnetic resonance spectroscopy (NMR), photon correlation spectroscopy (PCS), IR, surface plasma resonance (SPR), XPS, scanning probe microscopy (SPM), SEM, TEM, atomic absorption spectroscopy, elemental analysis, UV-vis, fluorescence spectroscopy, and the like.

Raman Spectroscopy

Raman Detectors

Various embodiments of the invention employ probe-conjugated reporter substrates in conjunction with known Raman spectroscopy techniques for a variety of applications, such as identifying and/or quantifying one or more analytes in a sample. In the practice of the present invention, the Raman spectrometer may be part of a detection unit designed to detect and quantify nanoparticles of the present invention by Raman spectroscopy. Methods for detection of Raman labeled analytes, for example nucleotides, using Raman spectroscopy are known in the art. (See, for example, U.S. Pat. Nos. 5,306,403; 6,002,471; 6,174,677). Variations on surface enhanced Raman spectroscopy (SERS), surface enhanced resonance Raman spectroscopy (SERRS) and coherent anti-Stokes Raman spectroscopy (CARS) have been disclosed.

A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams may be used. The excitation beam passes through confocal optics and a microscope objective, and is focused onto the flow path and/or the flow-through cell. The Raman emission light from the labeled nanoparticles is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics may be used as well as confocal optics. The Raman emission signal is detected by a Raman detector that includes an avalanche photodiode interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam may be spectrally purified with a bandpass filter (Corion) and may be focused on the flow path and/or flow-through cell using a 6× objective lens (Newport, Model L6×). The objective lens may be used to both excite the Raman-active organic compounds of the nanoparticles and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) may be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors may be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art may be used for detection of the nanoparticles of the present invention, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In one embodiment of the invention, an apparatus used in performing the invention methods is described with reference to FIGS. 11A and 11B. In apparatus 1000, Raman analyzer 1100 emits a beam of light 1220 from a light source 1120, to the surface of chip 1200, from which it is reflected back as scattered beam 1240. Spectroscope light detector 1160 receives scattered beam 1240, filtered through MEMS device 1250 and provides a signal representative of a spectrum of the scattered light to processor 1180. Raman analyzer 1100 may further include filter or prism 1140 to select a predetermined bandwidth of beam of light 1220 directed to chip 1200. On chip 1200, binding of a target biomolecule to a probe molecule, for example in a detection complex, causes a frequency shift in the spectrum of the scattered light beam 1240 detected by spectroscope light detector 1160 corresponding to a defined location on chip 1200, which detection is passed on to processor 1180. Two or more spectroscopes operating in parallel may be used for multiplex detection of signals from two or more locations on a chip surface (see FIG. 11B for example). As discussed herein, multiple subarrays on a chip can be scanned in a high throughput manner to effect rapid assay of, for example, the sequence of a polynucleotide, or to determine the presence of various biomolecules in a complex biological sample. FIG. 11B is an illustrative COIN array chip reader used in one aspect of the invention for detecting multiple signals. Such a reader includes parallel photodiode array sets 1300 to collect multiple spectra 1310 simultaneously from a sample 1320 on an array chip 1330 and may be used with an apparatus of FIG. 11A. As described above in FIG. 11A, the Raman analyzer 1100 may further include filter or prism 1140 (also shown as 1340 in FIG. 11B) to select a predetermined bandwidth of beam of light 1220 directed to chip 1200.

Micro-Electro-Mechanical Systems (MEMS)

In various embodiments of the invention, the chips and substrates may be incorporated into a larger apparatus and/or system. In certain embodiments, the apparatus may incorporate a micro-electro-mechanical system (MEMS). MEMS may be integrated systems comprising mechanical elements, sensors, actuators, and electronics. All of those components may be manufactured by known microfabrication techniques on a common chip, comprising a silicon-based or equivalent substrate (See, for example, Voldman et al., Ann. Rev. Biomed. Eng. 1:401-425, 1999). The sensor components of MEMS may be used to measure mechanical, thermal, biological, chemical, optical and/or magnetic phenomena. The electronics may process the information from the sensors and control actuator components such as pumps, valves, heaters, coolers, and filters, thereby controlling the function of the MEMS.

The electronic components of MEMS may be fabricated using integrated circuit (IC) processes (for example, CMOS, Bipolar, or BICMOS processes). They may be patterned using photolithographic and etching methods known for computer chip manufacture. The micromechanical components may be fabricated using compatible “micromachining” processes that selectively etch away parts of the silicon wafer, or comparable substrate, or add new structural layers to form the mechanical and/or electromechanical components.

Basic techniques in MEMS manufacture include depositing thin films of material on a substrate, applying a patterned mask on top of the films by photolithographic imaging or other known lithographic methods, and selectively etching the films. A thin film may have a thickness in the range of a few nanometers to 100 micrometers. Deposition techniques of use may include chemical procedures such as chemical vapor deposition (CVD), electrodeposition, epitaxy and thermal oxidation and physical procedures like physical vapor deposition (PVD) and casting. Methods for manufacture of nanoelectromechanical systems may be used for certain embodiments of the invention. (See, for example, Craighead, Science 290: 1532-36,2000.)

In some embodiments of the invention, the array or subarrays on a chip may be connected to various fluid filled compartments, such as microfluidic channels, nanochannels and/or microchannels. These and other components of the apparatus may be formed as a single unit, for example in the form of a chip, as known in semiconductor chips and/or microcapillary or microfluidic chips.

Techniques for batch fabrication of chips may be well known in the fields of computer chip manufacture and/or microcapillary chip manufacture. Such chips may be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding with a flowable, optically clear material such as plastic or glass; photolithography and dry etching of silicon dioxide; electron beam lithography using polymethylmethacrylate resist to pattern an aluminum mask on a silicon dioxide substrate, followed by reactive ion etching. Methods for manufacture of nanoelectromechanical systems may be used for certain embodiments of the invention. (See, for example, Craighead, Science 290:1532-36, 2000.) Various forms of microfabricated chips may be commercially available from, for example, Caliper Technologies Inc. (Mountain View, Calif.) and ACLARA BioSciences Inc. (Mountain View, Calif.).

In certain embodiments of the invention, part or all of the apparatus may be selected to be transparent to electromagnetic radiation at the excitation and emission frequencies used for Raman spectroscopy, such as glass, silicon, quartz or any other optically clear material. For fluid-filled compartments that may be exposed to various analytes, such as proteins, peptides, nucleic acids, nucleotides and the like, the surfaces exposed to such molecules may be modified by coating, for example to transform a surface from a hydrophobic to a hydrophilic surface and/or to decrease adsorption of molecules to a surface. Surface modification of common chip materials such as glass, silicon, quartz and/or PDMS is known in the art (for example, U.S. Pat. No. 6,263,286). Such modifications may include, but may be not limited to, coating with commercially available capillary coatings (Supelco, Bellafonte, Pa.), silanes with various functional groups, such as polyethyleneoxide or acrylamide, or any other coating known in the art.

In certain aspects of the invention, a system for detecting the nanoparticles of the present invention includes an information processing system. An exemplary information processing system may incorporate a computer that includes a bus for communicating information and a processor for processing information. In certain examples, the processor is selected from the Pentium® family of processors, including without limitation the Pentium® II family, the Pentium® III family and the Pentium® 4 family of processors available from Intel Corp. (Santa Clara, Calif.). In alternative embodiments of the invention, the processor may be a Celeron®, an Itanium®, or a Pentium Xeon® processor (Intel Corp., Santa Clara, Calif.). In various other embodiments of the invention, the processor may be based on Intel® architecture, such as Intel® IA-32 or Intel® IA-64 architecture. Alternatively, other processors may be used. The information processing and control system may further include any peripheral devices known in the art, such as memory, display, keyboard and/or other devices.

In particular examples, the detection unit may be operably coupled to the information processing system. Data from the detection unit may be processed by the processor and data stored in memory. Data on emission profiles for various Raman labels may also be stored in memory. The processor may compare the emission spectra from composite organic-inorganic nanoparticles in the flow path and/or flow-through cell to identify the Raman-active organic compound. The processor may analyze the data from the detection unit to determine, for example, the sequence of a polynucleotide bound by a probe of the nanoparticles of the present invention. The information processing system may also perform standard procedures such as subtraction of background signals

While certain methods of the present invention may be performed under the control of a programmed processor, in alternative embodiments of the invention, the methods may be fully or partially implemented by any programmable or hardcoded logic, such as Field Programmable Gate Arrays (FPGAs), TTL logic, or Application Specific Integrated Circuits (ASICs). Additionally, the disclosed methods may be performed by any combination of programmed general purpose computer components and/or custom hardware components.

Following the data gathering operation, the data will typically be reported to a data analysis operation. To facilitate the analysis operation, the data obtained by the detection unit will typically be analyzed using a digital computer such as that described above. Typically, the computer will be appropriately programmed for receipt and storage of the data from the detection unit as well as for analysis and reporting of the data gathered.

In certain embodiments of the invention, custom designed software packages may be used to analyze the data obtained from the detection unit. In alternative embodiments of the invention, data analysis may be performed, using an information processing system and publicly available software packages.

COIN beads used in invention methods are about 1 μm in diameter and include two or more invention COINs or clusters of COIN nanoparticles embedded and held together within a polymeric microsphere. Methods for making COIN beads will now be discussed. The structural features are a) a structural framework formed by polymerized organic compounds; b) multiple COINs embedded in a micro-sized particle; and c) a surface with suitable functional groups for attachment of desired molecules, such as linkers, probes, and the like. Such microspheres produce stronger and more consistent SERS signals than individual COINs or nanoparticle clusters or aggregates. The polymer coating of the large microsphere may also provide sufficient surface areas for attachment of biomolecules, such as probes. Several methods for producing COIN beads for use in the invention methods are set forth below.

Inclusion method This approach employs the well-established emulsion polymerization technique for preparing uniform latex microspheres, except that COIN particles are introduced into the micelles before polymerization is initiated. As shown in the flow chart of FIG. 11, this aspect of the invention methods involves the following steps: 1) Micelles of desired dimensions are first prepared by homogenization of water with surfactants (for example octanol). 2) COIN particles are introduced along with a hydrophobic agent (for example SDS). The latter facilitates the transport of COIN particles into the interior of micelles. 3) Micelles are protected against aggregation with a stabilizing agent (for example casein). 4) Monomers (for example styrene or methyl methacrylate) are introduced. 5) Finally, a free radical initiator (for example peroxide or persulfate) is used to start the polymerization to produce COIN embedded latex microspheres.

An important refinement of the above approach is to use clusters of COIN particles that have been embedded within a solid organic polymer bead to form a microsphere. The polymer may prevent direct contact between nanoparticle clusters or COIN particles in the micelles and in the final product (COIN bead). Furthermore, the number of COIN clusters or COIN labels in a microsphere may be adjusted by varying the polymer thickness in the interstices of the microsphere. The polymer material of the microsphere is not needed for signal generation, the function of the polymer being structural.

The COIN beads are about 1 micron to about 5 microns in average diameter and may operate as a functional unit having a structure comprising many individual COIN particles held together by the structural polymer of the microsphere. Thus, within a single microsphere are several COIN label or COIN particles embedded in the structural polymer, which is the main inner and outer structural material of the bead. The structural polymer also functions as a surface for attaching linkers, or can be functionalized for attachment of probes. Since a COIN comprises a cluster of primary metal particles with at least one Raman-active organic compound adsorbed on the metal particles, the polymer of the COIN bead for the most part does not come into contact with and hence does not attenuate Raman-activity of the Raman-active organic compounds that are trapped as they were adsorbed during colloid formation in the junctions of the primary metal particles or embedded in the metal crystals of the COIN structure. Those Raman-active organic molecules on the periphery of the COIN that may come into contact with the structural polymer of the microsphere have reduced effect as Raman-active molecules.

Soak-in method Another method for making the COIN beads used in the invention methods utilizes the following steps. Polymer beads are formed by emulsion polymerization. The polymer beads are subjected to an organic solvent, such as CHCB/Butanol, which causes the beads to swell such that pores of the polymer bead become enlarged. COIN particles are contacted with the swollen polymer beads, allowing the COIN particles to diffuse inside via the swollen pores. Changing the liquid phase to an aqueous phase causes the pores of the bead to close, embedding the COIN particles within the polymer beads. For example, 1) Styrene monomers may be co-polymerized with divinylstyrene and acrylic acid to form uniformly sized beads through emulsion polymerization. 2) The beads are swollen with organic solvents such as chloroform/ butanol, and a set of COIN particles is introduced at a ratio sufficient to cause the COIN particles to diffuse into the swollen bead. 3) The beads are then placed in a non-solvent to shrink the beads so that the COIN particles are trapped inside to form stable, uniform COIN beads. The COIN beads may be functionalized with probes, such an antibodies, to yield probe labeled COIN beads, which can be used in the place of probe-labeled COINs in the invention methods.

Build-in method Yet another method for making the COIN beads used in the invention methods includes the following steps. In this method, microspheric polymer beads are obtained first and are placed in contact with Raman active organic molecules and silver colloids in organic solvents. Under this condition, the pores of the beads are enlarged enough to allow the Raman active molecules and silver colloids to diffuse inside the swollen polymer beads. Then COIN clusters are formed inside the microspheres when silver colloids encounter one another in the presence of organic Raman labels. Heat and light may be used to accelerate aggregation and fusion of silver particles. Finally, the liquid phase is changed to aqueous phase, to yield COIN beads, which may be functionalized for attachment of probe molecules as described above. For example, 1) styrene monomers may be co-polymerized with divinylstyrene and acrylic acid to form uniformly sized beads through emulsion polymerization. 2) The beads are then swelled with organic solvents such as chloroform/butanol, and a set of Raman-active molecules (for example 8-aza-adenine and N-benzoyladenine) at a certain ratio is introduced so that the molecules diffuse into the swollen bead. A silver colloid suspension in the same solvent is then mixed with the beads to form silver particle-encapsulated beads. 3) The solvent is then switched to one that shrinks the beads so that the Raman labels and silver particles are trapped inside. The process may be controlled so that the silver particles will contact one another with Raman molecules in the junction, forming COIN particles inside the beads. When medium size silver colloids such as 60 nm are used, Raman labels may be added separately (before or after silver addition) to induce colloid aggregation (formation of COINs) inside the beads. When 1-10 nm colloids are used, the Raman -active organic compounds may be added together. Then light or heat may be used to induce the formation of COINs particles inside the microspheres.

Build-out method Yet another method for making the COIN beads used in the invention methods includes the following steps. A solid core is used first as a support for attachment of COIN particles. The core may be metal (gold and silver), inorganic (alumina, hematite and silica) or organic (polystyrene, latex) particles. Electrostatic attraction, van der Waals forces, and/or covalent binding may induce attachment of COIN particles to the core particle. After the attachment, the assembly may be coated and filled in with a polymer material to stabilize the structure and at the same time to provide a surface with functional groups. Multiple layers of COIN particles may be built based on the above procedure. The dimension of the COIN beads so produced may be controlled by the size of the core and the number of COIN-containing layers. For example, 1) positively charged Latex particles of 0.5 μm are mixed with negatively charged COIN particles, 2) the Latex-COIN complex is coated with a cross-linkable polymer such as poly-acrylic acid. 3) The polymer coating is cross-linked with linker molecules such as lysine to form an insoluble shell. Remaining (unreacted) carboxylic groups would serve as the functional groups for attachment of a second layer of COIN particles. Additional functional groups may also be introduced through co-polymerization or during the cross-link process.

A prerequisite for multiplex tests in a complex sample is to have a coding system that possesses identifiers for a large number of reactants in the sample. The primary variable that determines the achievable numbers of identifiers in currently known coding systems is, however, the physical dimension. Recently reported tagging techniques, based on surface-enhanced Raman scattering (SERS) of fluorescent dyes, show the possibility of developing chemical structure-based coding systems. The organic compound-assisted metal fusion (OCAM) method used to produce composite organic-inorganic nanoparticles (COIN) that are highly effective in generating SERS signals allows synthesis of COIN labels from a wide range of organic compounds to produce sufficient distinguishable COIN Raman signatures to assay any complex biological sample. Thus COIN particles may be used as a coding system for multiplex and amplification-free detection of bioanalytes at near single molecule levels.

COIN particles generate intrinsic SERS signal without additional reagents. Using the OCAMF-based COIN synthesis chemistry, it is possible to generate a large number of different COIN signatures by mixing a limited number of Raman labels for use in multiplex assays in different ratios and combinations. In a simplified scenario, the Raman spectrum of a sample labeled with COIN particles may be characterized by three parameters:

(a) peak position (designated as L), which depends on the chemical structure of Raman labels used and the umber of available labels,

(b) peak number (designated as M), which depends on the number of labels used together in a single COIN, and

(c) peak height (designated as i), which depends on the ranges of relative peak intensity.

The total number of possible distinguishable Raman signatures (designated as T) may be calculated from the following equation:

$$T = {\sum }_{k = 1}^{M} \frac{L !}{( L - k ) ! k !} P ( i , k )$$

where P(i, k)=i^{k}−i+1, being the intensity multiplier which represents the number of distinct Raman spectra that may be generated by combining k (k=1 to M) labels for a given i value. The multiple organic compounds may be mixed in various combinations, numbers and ratios to make the multiple distinguishable Raman signatures. It has been shown that spectral signatures having closely positioned peaks (15 cm⁻¹) may be resolved visually. Theoretically, over a million of Raman signatures may be made within the Raman shift range of 500-2000 cm⁻¹ by incorporating multiple organic molecules into COIN as Raman labels using the OCAMF-based COIN synthesis chemistry.

Thus, OCAMF chemistry allows incorporation of a wide range of Raman labels into metal colloids to perform parallel synthesis of a large number of COIN labels with distinguishable Raman signatures in a matter of hours by mixing several organic Raman-active compounds of different structures, mixtures, and ratios for use in the invention methods described herein.

The invention is further described by the following non-limiting example.

### EXAMPLE 1

Antibody-COIN conjugation: To conjugate COIN particles with antibodies, a direct adsorption method was used. A 500 μL solution containing 2 ng of a biotinylated anti-human IL-2 (anti-IL-2), or IL-8 antibody (anti-IL-8), in 1 mM Na₃Citrate (pH 9) was mixed with 500 μL of a COIN solution (using 8-aza-adenine or N-benzoyl-adenine as the Raman label); the resulting solution was incubated at room temperature for 1 hour, followed by adding 100 μL of PEG-400 (polyethylene glycol 400). The solution was incubated at room temperature for another 30 min before a 200 μL of 1% Tween-20 was added. The resulting solution was centrifuged at 2000×g for 10 min. After removing the supernatant, the pellet was resuspended in 1 mL solution (BSAT) containing 0.5% BSA, 0.1% Tween-20 and 1 mM Na₃Citrate. The solution was again centrifuged at 1000×g for 10 min to remove the supernatant. The BSAT washing procedure was repeated for a total of 3 times. The final pellet was resuspended in 700 μL of Diluting Solution (0.5% BSA, 1×PBS, 0.05% Tween-20). The Raman activity of a conjugated COIN label sample was measured and adjusted to a specific activity of about 500 photon counts (from main peak) per μL per 10 seconds using a Raman microscope that generated about 600 counts from methanol at 1040 cm⁻¹ for a 10 second collection time.

Immuno sandwich assays Xenobind™ Aldehyde slides (Xenopore Inc., NJ, USA) were used as substrates for immuno sandwich assays; before being used, wells on a slide were prepared by overlaying a slab of cured poly(dimethyl siloxane) (PDMS) elastomer of 1 mm thickness. Holes approximately, 5 mm in diameter were punched into the PDMS slab. To immobilize capture antibodies, 50 μL of an antibody (9 μg/mL) in 0.33×PBS was added to wells and the slide was incubated in a humidity chamber at 37° C. for 2 hours. After removing free antibodies, 50 μL of 1% BSA in a 10 mM glycine solution was added to the wells to inactivate the aldehyde groups on the slide. The slide was incubated at 37° C. for another 1 hour before the wells were washed 4 times, each with 50 μL PBST washing solution (1×PBS, supplemented with 0.05% Tween-20).

Antigen binding and detection antibody binding (antibody-COIN conjugate binding) were carried out following instructions from the antibody supplier (BD Biosciences). After removing the unbound conjugates, the wells were washed 4 times, each with 50 μL of washing solution. Finally, 30 μL of washing solution was added to wells before competitive binding. To demonstrate competitive binding, interleukin-2 protein (IL-2, 10 ng/mL) may be added to wells with anti-IL-2 capture antibody; anti-IL-2 antibody-coated COIN particles are used to bind to the captured IL-2 molecules in the binding complexes. After washing the wells with buffer, samples containing different amounts of IL-2 were added separately to the wells. The solutions containing released COINs from wells were detected for COIN signals with a Raman scope.

Although the invention has been described with reference to the above example, it will be understood that modifications and variations are encompassed within the spirit and scope of the invention. Accordingly, the invention is limited only by the following claims.

What is claimed is:

1. A system for sequencing a polynucleotide comprising:
one or more subsets of a first probe set, wherein a member of the first probe set comprises one or more probes and at least one label to produce distinguishable first and second optical signatures, wherein the first optical signature indicates attachment orientation of the probes within the first probe set and the second optical signature is a Raman signature associated with a known probe sequence of the member within a subset of the first probe set, and
one or more subsets of a second probe set, wherein a member of the second probe set comprises one or more probes and at least one label to produce distinguishable third and fourth optical signatures, wherein the third optical signature indicates an attachment orientation of the probes to the label that is opposite to that of the first probe set and the fourth optical signature is a Raman signature associated with a known sequence of the oligonucleotides of the member within a subset of the second probe set,
wherein the probe sequence of a member of a probe set is unique to the member within a respective probe set.

2. The system of claim 1, further comprising one or more subsets of a third probe set, wherein a member of the third probe set is unlabelled, comprises a probe of at least about 3 nucleotides, and forms a phosphodiester bond with a member of the first probe set.

3. The system of claim 1, wherein the probe sequences in the probe sets have a fixed length

4. The system of claim 1, wherein the first and third optical signatures are fluorescent and the second and fourth optical signatures are produced by COIN labels.

5. The system of claim 4, wherein the COIN labels in a probe set produce 100 or more distinguishable Raman signatures.

6. The system of claim 4, wherein the COIN labels of a probe set are COIN beads with the probe sequence conjugated to the exterior of the bead.

7. The system of claim 6, wherein the COIN beads have an average diameter in the range from about 0.1 micron to about 10 microns.

8. An array comprising:
a substrate comprising two or more fixed locations with surface attached coupling agents for binding to a reporter substrate conjugated to a probe; and
one or more adhere surfaces comprising an inorganic material overlaying the substrate,
wherein the adhere surfaces are modified by the surface attached coupling agents.

9. The array of claim 8, wherein the adhere surfaces comprise pads of an inorganic material selected from gold, silica, plastic, aluminum oxide, or platinum.

10. The array of claim 9, further comprising a protection layer overlaying the substrate between the fixed locations.

11. The array of claim 10, wherein the protection layer is a coating of a polyethylene glycol, a carbohydrate, a protein or a mixture thereof.

12. The array of claim 9, wherein the pads are electrically conductive.

13. The array of claim 9, wherein the pads are from about 10 nm to less than 100 microns in largest dimension.

14. The array of claim 8, wherein the coupling agents are selected from a thiol, a silane, protein G, protein A, poly(A), poly(T), streptavidin, biotin, antibodies, antigen, lectin, or carbohydrate.

15. The array of claim 8, wherein a single adhere surface is uniform.

16. The array of claim 15, wherein the coupling agents are selected from a thiol, a silane, protein G, protein A, poly(A), poly(T), streptavidin, biotin, antibodies, antigen, lectin, or carbohydrate.

17. The array of claim 8, wherein at least one array is located on the surface of a chip.

18. The array of claim 8, wherein the array is flexible.

19. A method for assaying a biological sample comprising at least one biomolecule, comprising:
a) contacting under conditions suitable to promote specific binding to form detection complexes between:
i) an array comprising a substrate comprising two or more fixed locations with surface attached coupling agents for binding to a reporter substrate conjugated to a probe; and one or more adhere surfaces comprising an inorganic material overlaying the substrate, wherein the adhere surfaces are modified by the surface attached coupling agents;
ii) a probe-conjugated substrate reporter comprising a label that produces a Raman signature attached to a probe molecule selected to bind specifically with the coupling agent attached to the fixed locations, wherein the probe molecule is selected from a thiol, a silane, protein G, protein A, poly(dA), poly(dT), streptavidin, or biotin, antibody, antigen, lectin, or carbohydrate; and
iii) a biological sample, wherein one or more biomolecules in the sample are prelabeled with a probe conjugate comprising a moiety that binds specifically with a known biomolecule conjugated to a label comprising one or more distinguishable Raman-active or fluorescent organic compounds;
b) detecting multiplex optical signals produced by detection complexes comprising a probe conjugate, a biomolecule and a probe-conjugated substrate reporter formed at one or more fixed locations of the array; and
c) determining the presence of one or more biomolecules at the fixed locations by associating the presence of a distinguishable Raman-active or fluorescent organic compound with the presence of the known biomolecule.

20. The method of claim 19, wherein the probe conjugate is a member of a set wherein a member of the set binds specifically to a known biomolecule and produces a distinguishable Raman-active signature associated with the biomolecule to which the member binds.

21. The method of claim 19, wherein the detection involves scanning the array to detect optical signals from detection complexes formed at two or more fixed locations of the array.

22. The method of claim 21, wherein the scanning of the arrays is performed in parallel.

22. The method of claim 20, wherein the probe-conjugated reporter substrate is a COIN label conjugated to one or more probes that bind specifically with the analyte.

23. The method of claim 22, wherein the probes comprise nucleotide sequences.

24. A method for assaying a biological sample comprising at least one biomolecule, comprising:
contacting an array under conditions suitable to promote formation of one more complexes between:
i) a probe-conjugated substrate reporter comprising a substrate reporter that produces a Raman signature conjugated to a first probe molecule that binds specifically with a known biomolecule, wherein the substrate-reporter comprises a coupling agent that forms a specific binding pair with the surface attached coupling agent attached to the adhere surface of the array, and
ii) a biological sample comprising one or more target biomolecules,
contacting the one or complexes formed in b) with a probe-conjugate comprising a second probe moiety that binds specifically with the known biomolecule and a distinguishable label Raman-active or fluorescent label, to form a detection complex at a fixed location;
detecting simultaneous optical signatures of detection complex formed at the fixed location of the array; and
d) determining presence of the known biomolecule at the fixed location by associating the optical signature of the label with the presence of the known biomolecule in the sample.

25. The method of claim 24, wherein one or both of the substrate reporter and the label comprise a COIN.

26. The method of claim 25, wherein the probe-conjugate is a member of a set, wherein binding specificity of a member of the first set is unique within the set and the label comprises one or more COINs that produce a distinguishable Raman signature associated with the biomolecule to which the member binds specifically.

27. The method of claim 24, wherein the label is Raman-active.

28. The method of claim 27, wherein the detecting uses parallel spectroscopes to simultaneously detect Raman signatures from two or more of the arrays on a chip.

29. The method of claim 27, wherein the probe-conjugate is a member of a set wherein binding specificity of the second probe is unique to the member within the set and the label comprises one or more COINs that produce a distinguishable Raman signature associated with the biomolecule to which the member binds specifically.

30. The method of claim 28, wherein the second probes in the set comprise antibodies.

31. A method for sequencing a target polynucleotide in a sample comprising:
a) contacting the sample containing the target polynucleotide with one or more subsets of the first probe set and one or more subsets of the third probe set under conditions suitable to result in specific hybridization of complementary nucleotide sequences, thereby forming hybridization complexes;
b) contacting the hybridization complexes formed in a) with one or more subsets of the second probe set under conditions suitable to result in hybridization of complementary sequences to form an at least partially double stranded tag hybridization complex containing a member of the first probe set, a member of the second probe set and a member of the third probe set;
c) detecting in multiplex the presence of the first, second, third, and fourth optical signatures associated with the at least partially double stranded tag hybridization complex; and
d) determining the nucleic acid sequence of the target polynucleotide included in a double stranded portion of the at least partially double stranded tag hybridization complex from the detected optical signatures.

32. The method of claim 31, wherein the method further comprises, prior to (b, ligating the probe sequences in the first and second probe sets under suitable ligation conditions to form the set of hybridization complexes.

33. The method of claim 32, wherein the contacting and the ligating steps are repeated under cycling conditions until members of the first probe set and the second probe set are substantially depleted from the sample.

34. A method for sequencing a target polynucleotide in a sample comprising:
a) contacting the sample containing the target polynucleotide with a subset of a first probe set and a subset of a third probe set under conditions suitable to result in specific hybridization of complementary nucleotide sequences, thereby forming hybridization complexes, wherein the sample is contacted with one or more subsets of a first probe set, wherein a member of the first probe set comprises one or more probes and at least one label to produce distinguishable first and second optical signatures, wherein the first optical signature indicates attachment orientation of the probes within the first probe set and the second optical signature is a Raman signature associated with a known probe sequence of the member within a subset of the first probe set, and one or more subsets of a second probe set, wherein a member of the second probe set comprises one or more probes and at least one label to produce distinguishable third and fourth optical signatures, wherein the third optical signature indicates an attachment orientation of the probes to the label that is opposite to that of the first probe set and the fourth optical signature is a Raman signature associated with a known sequence of the oligonucleotides of the member within a subset of the second probe set, wherein the probe sequence of a member of a probe set is unique to the member within a respective probe set;
b) contacting the hybridization complexes formed in a) with a subset of the second probe set under conditions suitable to result in hybridization of complementary sequences to form an at least partially double stranded tag hybridization complex containing a member of the first probe set, a member of the second probe set and a member of the third probe set;
c) detecting in multiplex presence of the first, second, third, and fourth optical signatures associated with the at least partially double stranded tag hybridization complex; and
d) determining the nucleic acid sequence of the target polynucleotide included in a double stranded portion of the at least partially double stranded tag hybridization complex from the detected optical signatures.

35. The method of claim 34, wherein the sample is contacted with the first probe set and the third probe set simultaneously.

36. The method of claim 34, wherein the labels comprise two or more COIN particles embedded within a polymeric bead.

37. The method of claim 36, wherein one or more of the nucleotide sequences is attached to the exterior of the polymeric bead.

38. The method of claim 34, wherein sequencing is performed using two or more arrays contained on a chip.

39. The method of claim 34, wherein two or more miniature spectroscopes operating in parallel are used for multiplex detection of the Raman signatures.

40. A Raman analyzer comprising:
a) a light source to emit a beam of light onto a chip surface;
b) at least one spectroscope to detect light from the beam that is scattered off the surface of the chip, the spectroscope to provide signals representative of one or more Raman signatures represented in the scattered light; and
c) a processor programmed to analyze simultaneous optical signatures resulting from a complex formed at a location of an array on the chip between:
i) a probe-conjugated substrate reporter comprising a substrate reporter that produces a Raman signature conjugated to a first probe molecule that binds specifically with a known biomolecule, wherein the substrate-reporter comprises a coupling agent that forms a specific binding pair with the surface attached coupling agent attached to the adhere surface of the array, and
ii) a biological sample comprising one or more target biomolecules, wherein contacting the one or complexes formed with a probe-conjugate comprising a second probe moiety that binds specifically with the known biomolecule and a distinguishable label Raman-active or fluorescent label, to form a detection complex at a fixed location; to determine binding of a target analyte by a change in the beam that is scattered off the surface of the chip, thereby determining the presence of the known biomolecule at the fixed location by associating the optical signature of the label with the presence of the known biomolecule in the sample.

41. An apparatus of claim 40, further comprising a filter to select a predetermined bandwidth of the beam of light emitted by the light source.

42. An apparatus of claim 40, wherein two or more spectroscopes operate in parallel to detect the scattered light.

43. An apparatus of claim 41, further comprising a MEMS component.
